(19) United States
(12) Patent Application Publication
(10) Pub. No.: US 2008/0108568 A1
(43) Pub. Date: May 8, 2008

(54) COMPOUNDS FOR IMPROVING LEARNING AND MEMORY

(75) Inventors: Dietrich A. Stephan, Phoenix, AZ (US); Matthew J. Huentelman, Phoenix, AZ (US); Andreas Papassotiropoulos, Zurich (CH); Dominique J.-F. de Quervain, Zurich (CH)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: TGEN; University of Zurich

(21) Appl. No.: 11/837,326

(22) Filed: Aug. 10, 2007

Related U.S. Application Data
(60) Provisional application No. 60/837,030, filed on Aug. 10, 2006.
(60) Provisional application No. 60/917,476, filed on May 11, 2007.

(51) Int. Cl. A61K 31/551; A61K 31/167; A61K 31/472; A61K 31/4985; A61K 38/16; C12Q 1/68; A61P 25/28; A61K 38/10; A61K 31/505; A61K 31/519

(52) U.S. Cl. 514/12; 435/6; 514/13; 514/14; 514/218; 514/250; 514/252.16; 514/275; 514/307; 514/613

(57) ABSTRACT

The present invention provides a method for improving learning and memory in a subject by administering a therapeutically effective amount of a compound of Formula I: 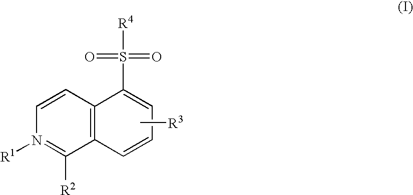
or of Formula II: 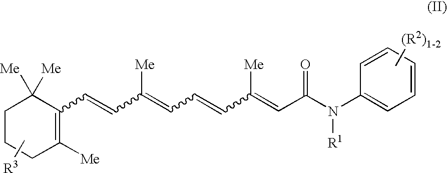 or of Formula III:
(R¹)ₓ-Ser-Ile-Tyr-Arg-Arg-Gly-Ala-Arg-Arg-Trp-Arg-Lys-Leu —(R²)_{y} (III).

### CROSS-REFERENCES TO RELATED APPLICATIONS

The present application claims priority to U.S. Ser. No. 60/837,030, filed Aug. 10, 2006, and U.S. Ser. No. 60/917,476, filed May 11, 2007, herein incorporated by reference in their entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

Human memory is a polygenic cognitive trait. Heritability estimates of ˜50% suggest that naturally occurring genetic variability has an important impact on this fundamental brain function. Recent candidate gene association studies have identified some genetic variations with significant impact on human memory capacity. However, the success of these studies depends upon preexisting information, which limits their potential to identify unrecognized genes and molecular pathways.

Recent advances in the development of high-density genotyping platforms have enabled the identification of some of the genes responsible for episodic and long-term memory performance (Papassotiropoulos et al. Science 2006, 314, 475). However, there is still no treatment available for subjects suffering from deteriorating episodic or long-term memory. Surprisingly, this invention meets this and other needs, such as improving learning and memory.

### BRIEF SUMMARY OF THE INVENTION

In one aspect, the present invention provides a method for improving learning and memory in a subject, the method comprising administering to a patient in need thereof, a therapeutically effective amount of a compound of Formula I: 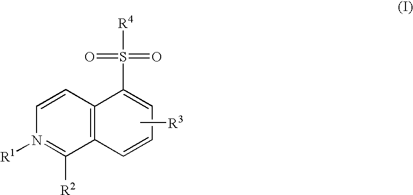
wherein R¹ is absent or is a member selected from the group consisting of hydrogen and C₁₋₆alkyl; R² is a member selected from the group consisting of hydrogen, hydroxy and halogen; R³ is a member selected from the group consisting of hydrogen and C₁₋₆alkyl; R⁴ is an N-linked heterocyclic ring system having from 5 to 8 ring members and two N ring heteroatoms, substituted with 0-3 R⁵ groups, wherein each R⁵ is independently a member selected from the group consisting of hydrogen, C₁₋₆alkyl, benzyl and phenyl; and prodrugs, salts, hydrates and solvates thereof.

In another aspect, the present invention provides a method for improving neural plasticity in a subject, the method comprising administering to a patient in need thereof, a therapeutically effective amount of a compound of Formula I: 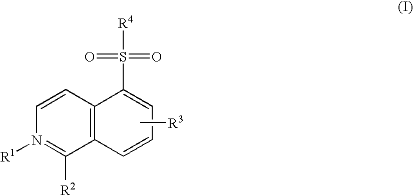
wherein R¹ is absent or is a member selected from the group consisting of hydrogen and C₁₋₆alkyl; R² is a member selected from the group consisting of hydrogen, hydroxy and halogen; R³ is a member selected from the group consisting of hydrogen and C₁₋₆alkyl; R⁴ is an N-linked heterocyclic ring system having from 5 to 8 ring members and two N ring heteroatoms, substituted with 0-3 R⁵ groups, wherein each R⁵ is independently a member selected from the group consisting of hydrogen, C₁₋₆alkyl, benzyl and phenyl; and prodrugs, salts, hydrates and solvates thereof.

In another aspect, the present invention provides a method for treating Alzheimer's disease in a subject, the method comprising administering to a patient in need thereof, a therapeutically effective amount of a compound of Formula I: 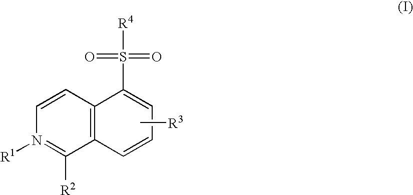
wherein R¹ is absent or is a member selected from the group consisting of hydrogen and C₁₋₆alkyl; R² is a member selected from the group consisting of hydrogen, hydroxy and halogen; R³ is a member selected from the group consisting of hydrogen and C₁₋₆alkyl; R⁴ is an N-linked heterocyclic ring system having from 5 to 8 ring members and two N ring heteroatoms, substituted with 0-3 R⁵ groups, wherein each R⁵ is independently a member selected from the group consisting of hydrogen, C₁₋₆alkyl, benzyl and phenyl; and prodrugs, salts, hydrates and solvates thereof.

In some embodiments, R⁴ is a 7-membered heterocyclic ring system. In other embodiments, the compound is of Formula Ia:

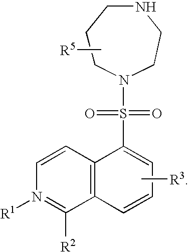

In other embodiments, the compound is:

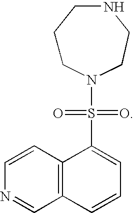

In still other embodiments, the compound is:

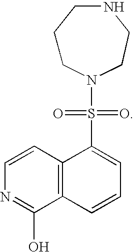

In another embodiment, the compound is the HCl salt. In some embodiments, the compound is:

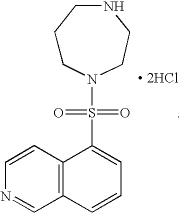

In a further embodiment, the compound is the hydrate. In another embodiment, the compound is: 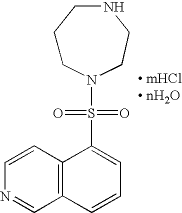
wherein m is 1 or 2; and n is from ½ to 3.

In one embodiment, the compound of Formula I is administered with a nitric oxide enhancing agent. In another embodiment, the nitric oxide enhancer is selected from the group consisting of a PDE5 inhibitor, a nitric oxide donor molecules, or a HMG Co A Reductase. In another embodiment, the nitric oxide enhancer is selected from the group consisting of Sildenafil, Tadalafil, Vardenafil, sodium nitroprusside, nitroglycerin, Atorvastatin, Simvastatin, Lovastatin, Fluvastatin, Pravastatin, Mevastatin, Pitavastatin, and Rosuvastatin. In another embodiment, the compound of Formula I and the nitric oxide enhancing agent are administered in together in the same composition. In another embodiment, the compound of Formula I and the nitric oxide enhancing agent are administered in together different compositions. In another embodiment, the compound of Formula I and the nitric oxide enhancing agent are administered at the same time. In another embodiment, the compound of Formula I and the nitric oxide enhancing agent are administered at different times.

In another aspect, the present invention provides a method for improving learning and memory in a subject, the method comprising administering to a patient in need thereof, a therapeutically effective amount of a compound of Formula II: 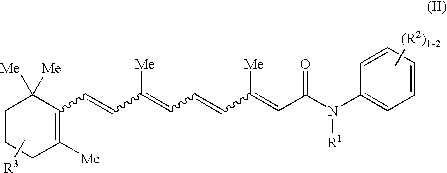
wherein R¹ is a member selected from the group consisting of hydrogen and C₁₋₆ alkyl; each R² is a member selected from the group consisting of hydrogen, C₁₋₆ alkyl, hydroxy and —O—C₁₋₆ alkyl; R³ is a member selected from the group consisting of hydrogen and C₁₋₆ alkyl; each [symbol] represents that the double bond to which it is attached is cis or trans; and prodrugs, salts, hydrates and solvates thereof.

In another aspect, the present invention provides a method for improving neural plasticity in a subject, the method comprising administering to a patient in need thereof, a therapeutically effective amount of a compound of Formula II: 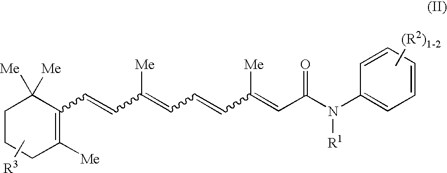
wherein R¹ is a member selected from the group consisting of hydrogen and C₁₋₆ alkyl; each R² is a member selected from the group consisting of hydrogen, C₁₋₆ alkyl, hydroxy and —O—C₁₋₆ alkyl; R³is a member selected from the group consisting of hydrogen and C₁₋₆ alkyl; each represents that the double bond to which it is attached is cis or trans; and prodrugs, salts, hydrates and solvates thereof.

In another aspect, the present invention provides a method for treating Alzheimer's disease in a subject, the method comprising administering to a patient in need thereof, a therapeutically effective amount of a compound of Formula II: 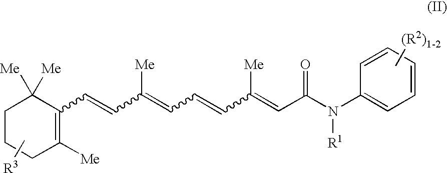
wherein R¹ is a member selected from the group consisting of hydrogen and C₁₋₆ alkyl; each R² is a member selected from the group consisting of hydrogen, C₁₋₆ alkyl, hydroxy and —O—C₁₋₆ alkyl; R³ is a member selected from the group consisting of hydrogen and C₁₋₆ alkyl; each [symbol] represents that the double bond to which it is attached is cis or trans; and prodrugs, salts, hydrates and solvates thereof.

In some embodiments, the compound is of Formula IIa:

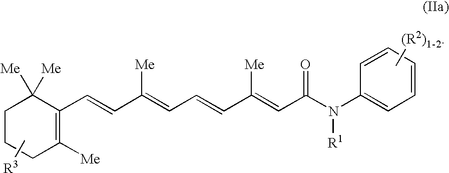

In other embodiments, the compound is:

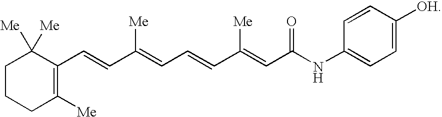

In another aspect, the present invention provides a method for improving learning and memory in a subject, the method comprising: administering to a patient in need thereof, a therapeutically effective amount of a compound of Formula III:
(R¹)ₓ—C—B-Tyr-Arg-Arg-A-A-Arg-Arg-Trp-Arg-Lys-B—(R²)_{y}
and conservatively modified variations thereof, in which: R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; A represents glycine or alanine; B represents isoleucine, leucine, methionine or valine; C represents serine or threonine; and x and y are independently selected and are equal to zero or one.

In another aspect, the present invention provides a method for improving neural plasticity in a subject, the method comprising: administering to a patient in need thereof, a therapeutically effective amount of a compound of Formula III:
(R¹)ₓ—C—B-Tyr-Arg-Arg-A-A-Arg-Arg-Trp-Arg-Lys-B—(R²)_{y}
and conservatively modified variations thereof, in which: R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; A represents glycine or alanine; B represents isoleucine, leucine, methionine or valine; C represents serine or threonine; and x and y are independently selected and are equal to zero or one.

In another aspect, the present invention provides a method for treating Alzheimer's disease in a subject, the method comprising: administering to a patient in need thereof, a therapeutically effective amount of a compound of Formula III:
(R¹)ₓ—C—B-Tyr-Arg-Arg-A-A-Arg-Arg-Trp-Arg-Lys-B—(R²)_{y}
and conservatively modified variations thereof, in which: R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; A represents glycine or alanine; B represents isoleucine, leucine, methionine or valine; C represents serine or threonine; and x and y are independently selected and are equal to zero or one.

In some embodiments, the method of the present invention comprises administering to a patient in need thereof, a therapeutically effective amount of a compound of Formula III:
(R¹)ₓ-Ser-Ile-Tyr-Arg-Arg-Gly-Ala-Arg-Arg-Trp-Arg-Lys-Leu —(R²)_{y}
and conservatively modified variations thereof, in which: R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; A represents glycine or alanine; B represents isoleucine, leucine, methionine or valine; C represents serine or threonine; and x and y are independently selected and are equal to zero or one.

In another embodiment, the present invention provides a method comprising administering to a patient in need thereof, a therapeutically effective amount of a compound of Formula III:
(R¹)ₓ-Ser-Ile-Tyr-Arg-Arg-Gly-Ala-Arg-Arg-Trp-Arg-Lys-Leu —(R²)_{y}
and conservatively modified variations thereof, in which: x and y are zero.

In another aspect, the present invention provides a method of identifying an increased risk of developing Alzheimer's disease in a subject, the method comprising the steps of obtaining a biologicial sample from a subject and identifying the presence or absence of the C-allele of SNP rs 17070145 in nucleic acid from the sample, wherein the presence of one or more copies of the C-allele indicates an increased risk in developing Alzheimer's disease as compared to subjects lacking the C-allele.

In one embodiment, the sample is blood. In another embodiment, the nucleic acid is DNA. In another embodiment, the presence or absence of the C-allele is identified using PCR.

### BRIEF DESCRIPTION OF THE DRAWINGS

FIG. 1 shows a table of inhibitor pharmacokinetics for fasudil, hydroxyfasudil, Y-27632 and H-1152P.

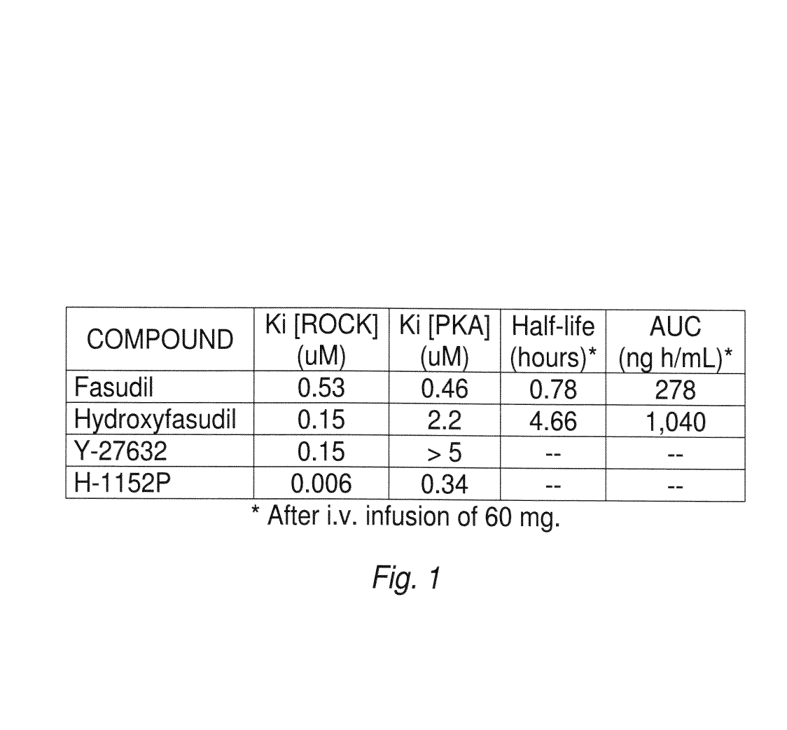

FIG. 2 shows a table of dose comparison data for fasudil in humans and hydroxyfasudil in rats. Oral Fasudil is typically administered to patients three times daily at doses between 40-80 mg each dose. Assuming a mean weight of 55 kg this equals a dose of between 0.7-1.4 mg per dosing or 2.1-4.2 mg per day.

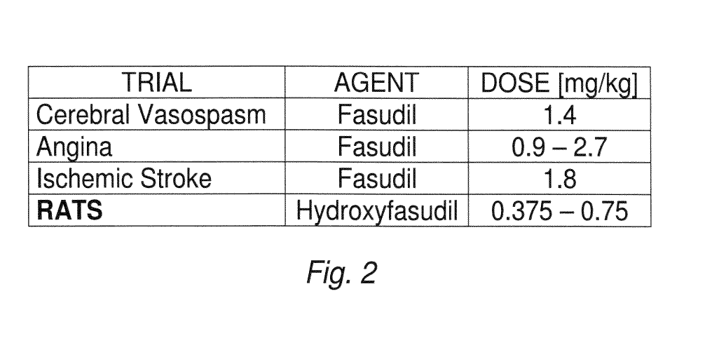

FIG. 3 shows a table for working memory incorrect following administration of hydroxyfasudil. The learning index is calculated by the following equation:

$$LearningIndex = \frac{{Errors}_{INITIAL} - {Errors}_{LATTER}}{{Errors}_{INITIAL}}$$

FIG. 4 shows a table for working memory correct following administration of hydroxyfasudil, the values calculated using the equation above for learning index.

FIG. 5 shows the administration of hydroxyfasudil improves working memory.

FIG. 6 shows an expression profile that reveals the alteration of “memory molecule” transcripts in the hippocampus of hydroxyfasudil treated animals. The tissue source is the entire hippocampus.

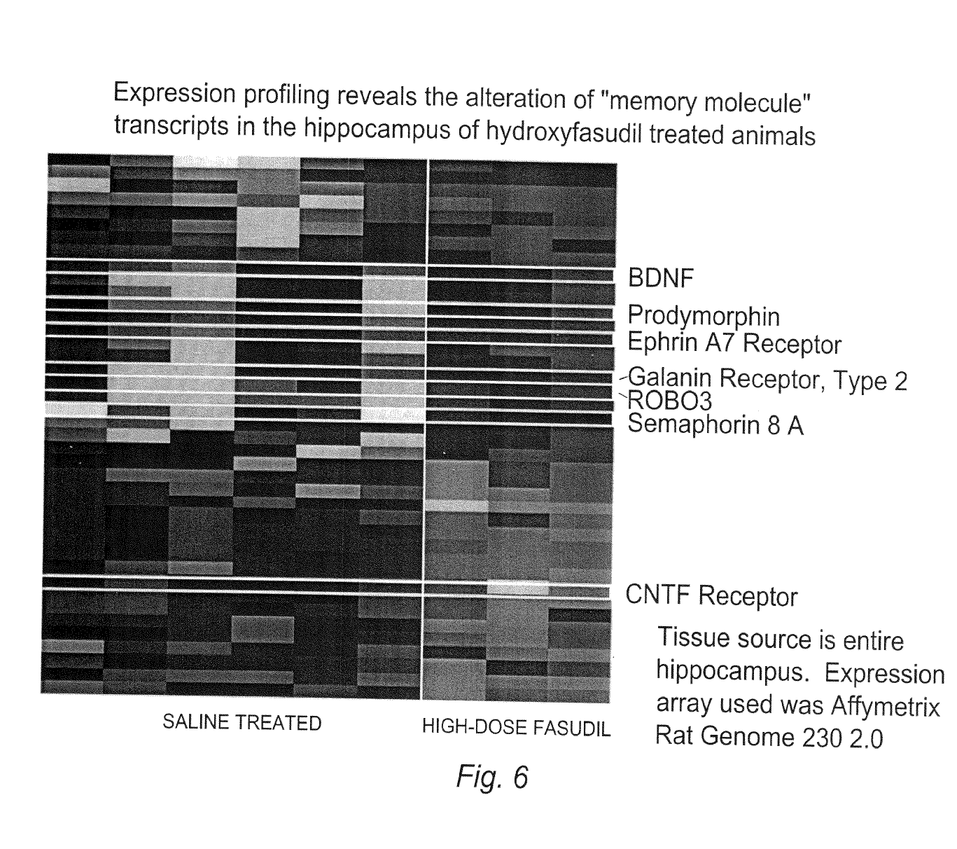

FIG. 7 shows fenretinide as improving working memory. There were 6 rats per group.

FIG. 8 shows a table of dose comparison data for fenretinide in humans and rats.

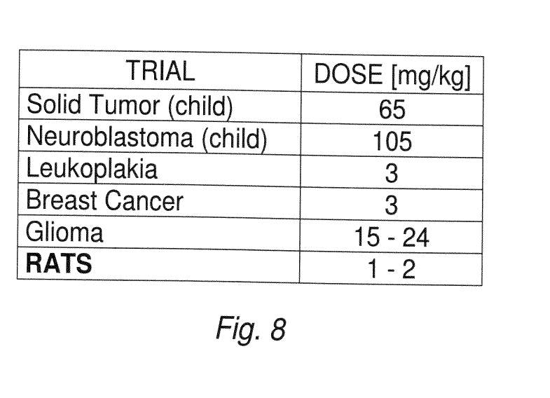

FIG. 9 shows KIBRA interacting pathways.

FIGS. 10 shows KIBRA-derived targets from the KIBRA-associated pathway and activator compounds or analogs (ceramide analogs).

FIG. 11A shows Fasudil improves reference memory performance as measured in the Morris water maze. Young and aged vehicle groups are indicated. Score is expressed in inches swam to locate the hidden platform in the Morris water maze. Data is collapsed across all five trials performed each day and is represented as mean ± S.D.

FIG. 11B shows Fasudil improves reference memory performance as measured in the Morris water maze. Young and aged vehicle groups are indicated. Score is expressed in inches swam to locate the hidden platform in the Morris water maze. Data is collapsed across by trial for all four days of testing and is represented as mean ± S.D.

FIG. 12 shows Fasudil improves memory performance.

FIG. 13 shows the association of KIBRA with Alzheimer's Disease, and that the most significantly associate haplotype block contains SNP rs1707014.

FIG. 14 shows in situ hybridization of the genetic target showing expression in the mouse hippocampus.

FIG. 15 shows a functional magnetic resonance imaging (fMRI) map showing non-carriers of the T allele with significantly increased brain activations compared to T allele carriers in the medial temporal lobe during memory retrieval.

FIG. 16 shows the administration of hydroxyfasudil improves working memory.

### DETAILED DESCRIPTION OF THE INVENTION

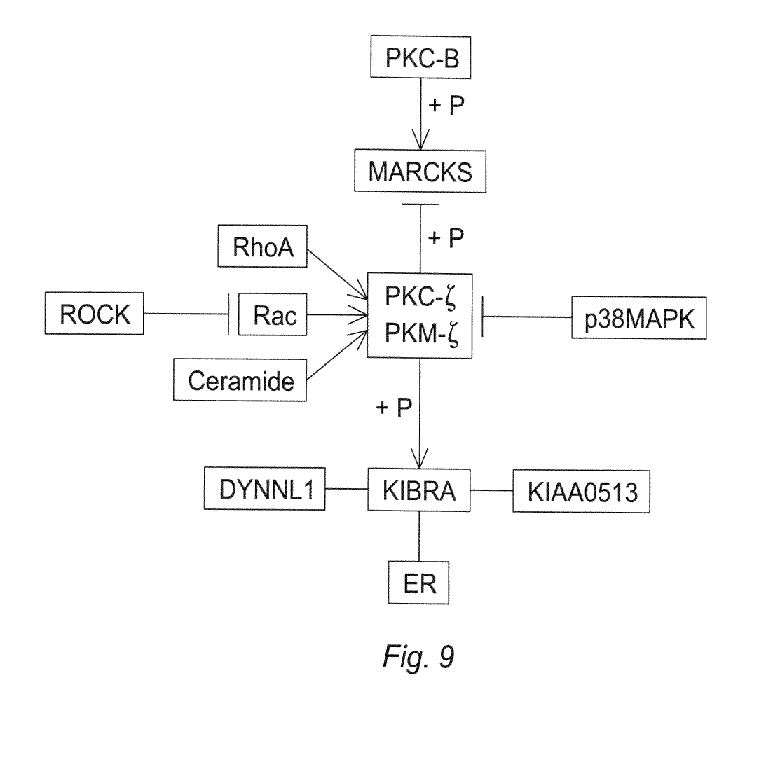

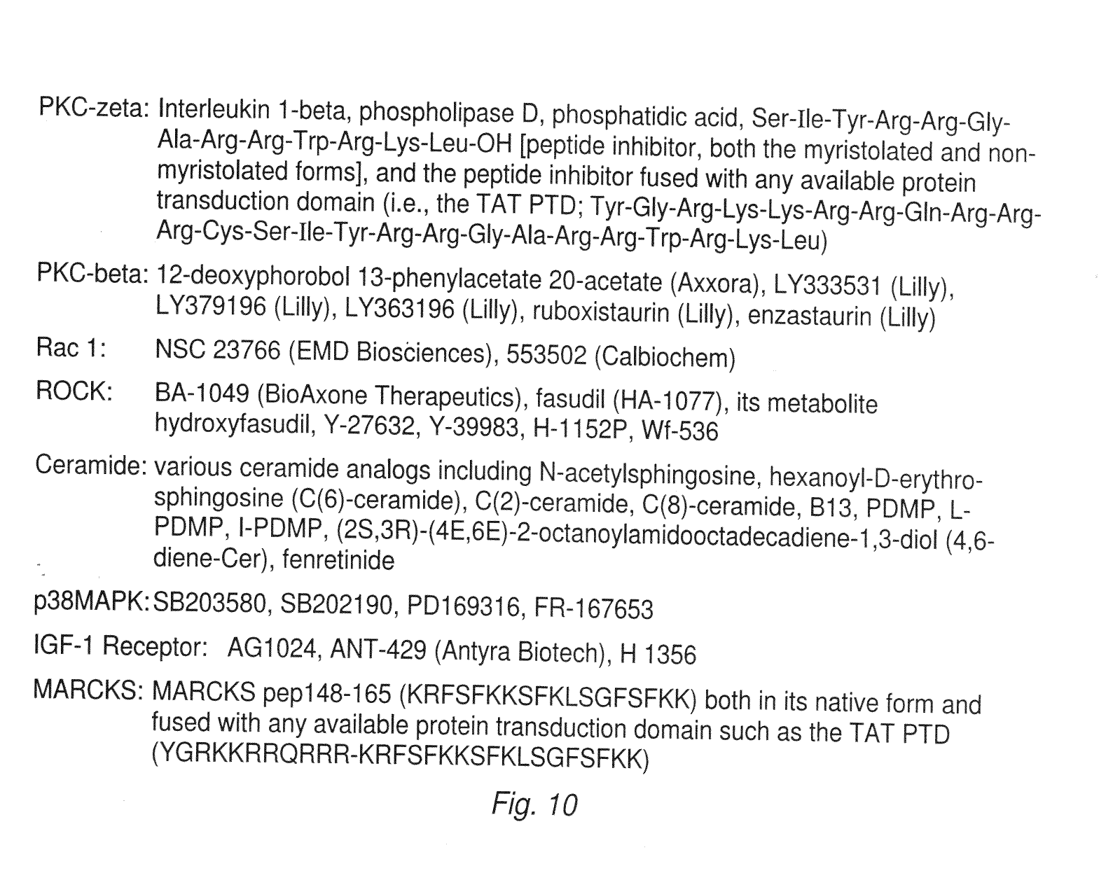

The present invention provides a new method for enhancing memory and learning. The effective enhancement of memory and learning is achieved by administering a drug that modulates KIBRA and KIBRA interacting pathways (upstream and downstream; see FIG. 9). Such drugs include fasudil, hydroxyfasudil, fenretinide, a PKZ-zeta peptide pseudo substrate, dimethylsphingosine, CVS-3989, D4476, AG1024, 648450, K252a, SB203580, C3 transferase, 553502, LY333531, ruboxistaurin, Go-6976, and other compounds listed in FIG. 10. Variants and derivatives of these compounds are also described herein.

Data presented herein also shows that the KIBRA gene, and in particular alleles associated with SNP rs17070145, indicate an increased risk of developing Alzheimer's disease. Subjects having at least one copy of the C-allele of this SNP show an increased risk of developing MCI and Alzheimer's disease as compared to subjects lacking the C-allele. Therefore, the invention provides a diagnostic test that indicates an increased risk in developing Alzheimer's disease. Patients having the C-allele can be monitored more closely for the disease state and can engage in prophylactic lifestyle changes and therapies, including medication, to prolong a disease free state, or avoid the disease altogether. Identification of this allele is also helpful for actual diagnosis of Alzheimer's disease, which is often considered a diagnosis of exclusion. Furthermore, the compounds described herein can be used not only to treat memory loss, which is a symptom of Alzheimer's disease, but can be used to treat a cause of Alzheimer disease and delay onset or prevent development of the disease (compare WO 2005/117896, which provides no mechanism or genetic link; see also Clin Neurophamacol 19:428 (1996)). Without being held to theory, it is thought that the KIBRA gene pathway is related to development of neurofibrillary tangles.

Perhaps the two most studied proteins linked to memory are PKC and cyclic AMP response element binding protein (CREB). PKC family members play a purported role in memory due to their overexpression in several key brain regions, their involvement in memory processes across several species, their age-related alterations in activity in humans correlated with spatial learning deficits, and finally the evidence that PKC inhibition impairs learning and memory (Micheau, J. & Riedel, G. Cell Mol Life Sci 55, 534-48 (1999); Pascale, A., et al. Mol Neurobiol 16, 49-62 (1998); Sun, M. K. & Alkon, D. L. Curr Drug Targets CNS Neurol Disord 4, 541-52 (2005); Birnbaum, S. G. et al. Science 306, 882-4 (2004); Etcheberrigaray, R. et al. Proc Natl Acad Sci U S A 101, 11141-6 (2004); Ruiz-Canada, C. et al. Neuron 42, 567-80 (2004)). Support for CREB as a memory-related gene include its defined role in long-term facilitation in the sea slug, Aplysia, and potentiation in rodents, the demonstration that the inducible disruption of CREB function blocks memory in mice, and exploration into compounds that alter CREB activity as memory enhancers (Josselyn, S. A. & Nguyen, P. V. Curr Drug Targets CNS Neurol Disord 4, 481-97 (2005); Carlezon, W. A., et al. Trends Neurosci 28, 436-45 (2005); Cooke, S. F. & Bliss, T. V. Curr Opin Investig Drugs 6, 25-34 (2005); Josselyn, S. A., Kida, S. & Silva, A. J. Neurobiol Learn Mem 82, 159-63 (2004); Martin, K. C. Neurobiol Learn Mem 78, 489-97 (2002); Lonze, B. E. & Ginty, D. D. Neuron 35, 605-23 (2002); Si, K., Lindquist, S. & Kandel, E. R Cell 115, 879-91 (2003); Chen, A. et al. Neuron 39, 655-69 (2003)). Additionally, there is mounting genetic evidence supporting the role of other proteins in memory including HTR2A, BDNF, and PKA (Alonso, M. et al. Learn Mem 12, 504-10 (2005); Bramham, C. R. & Messaoudi, E. Prog Neurobiol 76, 99-125 (2005); Papassotiropoulos, A. et al. Neuroreport 16, 839-42 (2005); de Quervain, D. J. et al. Nat Neurosci 6, 1141-2 (2003); Reynolds, C. A., et al. Neurobiol Aging 27, 150-4 (2006); Arnsten, A. F., et al. Trends Mol Med 11, 121-8 (2005); Quevedo, J. et al. Behav Brain Res 154, 339-43 (2004)).

KIBRA was recently identified in a yeast two hybrid screen as the binding partner for the human isoform of dendrin, a putative modulator of synaptic plasticity (Kremerskothen, J. et al., Biochem. Biophys. Res. Commun. 300, 862 (2003)). A truncated form, which was expressed in the hippocampus, lacks the first 223 aa and contains a C2-like domain, a glutamic acid-rich stretch and a protein kinase C (PKC) ζ-interacting domain (de Quervain, D. J. et al., Nat. Neurosci. 6, 1141 (2003)). PKC-ζ is involved in memory formation and in the consolidation of long-term potentiation (Bookheimer, S. Y. et al., N. Engl. J. Med. 343, 450 (2000); Milner, B. Clin. Neurosurg. 19, 421 (1972)). The C2-like domain of KIBRA is similar to the C2 domain of synaptotaginin, which is believed to function as the main Ca²⁺ sensor in synaptic vesicle exocytosis (Freedman, M. L. et al., Nat. Genet. 36, 388 (2004); Schacter, D. L. & Tulving E. Memory systems (MIT Press, Cambridge, 1994)). The memory-associated KIBRA haplotype block and SNP described herein map within the truncated KIBRA, which contains both the C2-like and the PKC-ζ-interacting domains. Taken together, the evidence from independent experiments in the present study suggests a role for KIBRA in normal human memory performance.

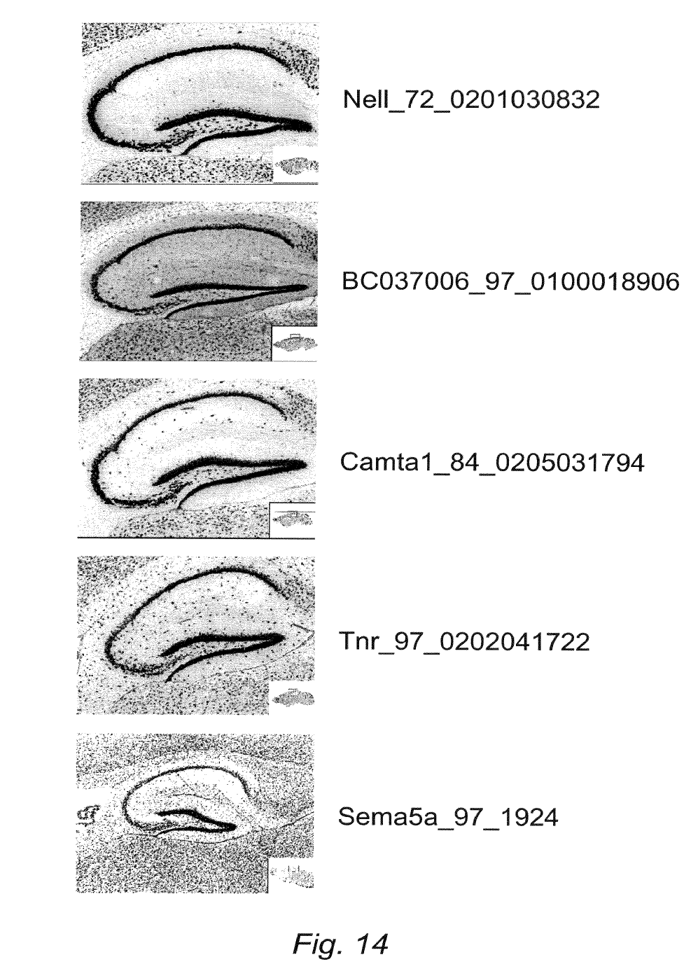

In addition to KIBRA, which has high expression high expression in brain, modulates Ca²⁺, is a PKC substrate, and a synaptic protein, there are several other genetic findings that have allowed the identification of RhoA/ROCK as a target in memory, and fasudil as a modulator to enhance memory, learning and cognition. CLSTN2 has high expression in brain, regulates Ca²⁺, and is a synaptic protein. CAMTA1 has high expression in brain, modulates Ca²⁺, and is a transcription factor. SEMA5A has high expression in the developing brain, and is involved in axonal guidance. TNR has high expression in the brain, is involved in the ECM, and assists in synapse maintenance. Finally, NELL2 also high expression in brain, assists in neuronal growth, and a mouse model (KO) shows enhanced LTP but impaired HPF-mediated learning. In addition, in situ hybridization of every one of the genetic targets shows expression in the mouse hippocampus (FIG. 14).

The fasudil target was successfully identified as being only one step away from the KIBRA protein in this pathway, and likely modulates pathway function upstream of KIBRA (FIG. 9). Compounds and pharmaceutical formulations of fasudil are described, e.g., in U.S. Pat. Nos. 4,678,783; 5,942,505; and 6,699,508, herein incorporated by reference in their entirety.

The significance of the RhoA/ROCK pathway in normal memory function as well as in Alzheimer's cognitive decline (and likely other amnestic disorders) cannot be understated. Many devastating disorders include memory loss as a primary clinical characteristic and in the case of these disorders the RhoA/ROCK pathway may play a role in their overall severity, progression, or pathology. Even minimal prolongation before memory loss onset would be beneficial to patients suffering from these disorders.

Pathologies or neuropathologies that would benefit from therapeutic and diagnostic applications of this invention include, for example, the following:

Diseases of central motor systems including degenerative conditions affecting the basal ganglia (Huntington's disease, Wilson's disease, striatonigral degeneration, corticobasal ganglionic degeneration), Tourette's syndrome, Parkinson's disease, progressive supranuclear palsy, progressive bulbar palsy, familial spastic paraplegia, spinomuscular atrophy, ALS and variants thereof, dentatorubral atrophy, olivo-pontocerebellar atrophy, paraneoplastic cerebellar degeneration, and dopamine toxicity;

Diseases affecting sensory neurons such as Friedreich's ataxia, diabetes, peripheral neuropathy, retinal neuronal degeneration;

Diseases of limbic and cortical systems such as cerebral amyloidosis, Pick's atrophy, Retts syndrome;

Neurodegenerative pathologies involving multiple neuronal systems and/or brainstem including Alzheimer's disease, AIDS-related dementia, Leigh's disease, diffuse Lewy body disease, epilepsy, multiple system atrophy, Guillain-Barre syndrome, lysosomal storage disorders such as lipofuscinosis, late-degenerative stages of Down's syndrome, Alper's disease, vertigo as result of CNS degeneration;

Pathologies associated with developmental retardation and learning impairments, and Down's syndrome, and oxidative stress induced neuronal death;

Pathologies arising with aging and chronic alcohol or drug abuse including, for example, with alcoholism the degeneration of neurons in locus coeruleus, cerebellum, cholinergic basal forebrain; with aging degeneration of cerebellar neurons and cortical neurons leading to cognitive and motor impairments; and with chronic amphetamine abuse degeneration of basal ganglia neurons leading to motor impairments;

Pathological changes resulting from focal trauma such as stroke, focal ischemia, vascular insufficiency, hypoxic-ischemic encephalopathy, hyperglycemia, hypoglycemia, closed head trauma, or direct trauma;

Pathologies arising as a negative side-effect of therapeutic drugs and treatments (e.g., degeneration of cingulate and entorhinal cortex neurons in response to anticonvulsant doses of antagonists of the NMDA class of glutamate receptor, chemotherapy, antibiotics, etc.).

Learning disabilities such as ADD, ADHD, dyslexia, dysgraphia, dyscalcula, dyspraxia, and information processing disorders.

I. Definitions

Memory systems can be classified broadly into four main types: episodic, semantic, working, and procedural (Hwang, D. Y. & Golby, A. J. Epilepsy Behav (2005); Yancey, S. W. & Phelps, E. A. J Clin Exp Neuropsychol 23, 32-48 (2001)). Episodic memory refers to a system that records and retrieves autobiographical information about experiences that occurred at a specific place and time. The semantic memory system stores general factual knowledge unrelated to place and time (e.g. the capital of Arizona). Working memory involves the temporary maintenance and usage of information while procedural memory is the action of learning skills that operate automatically and, typically, unconsciously. Episodic, semantic, and working memory are explicit (absolute) and declarative (explanatory) in nature while procedural memory can be either explicit or implicit, but is always nondeclarative (Tulving, E. Oxford University Press, New York, 1983); Budson, A. E., Price, B. H. Encyclopedia of Life Sciences (Macmillan, Nature Publishing Group, London, 2001); Budson, A. E. & Price, B. H. N Engl J Med 352, 692-9 (2005); Hwang, D. Y. & Golby, A. J Epilepsy Behav 8, 115-26 (2006)). See also Tsien, The Memory Code, Scientific American, Jun. 17, 2007.

Normal aging states and disease states that impair memory and/or learning include but are not limited to neurodegenerative disorders, head and brain trauma, genetic disorders, infectious disease, inflammatory disease, medication, drug and alcohol disorders, cancer, metabolic disorders, mental retardation, and learning and memory disorders, such as age related memory loss and age-associated memory impairment (AAMI), Alzheimer's disease, tauopathies, PTSD (post traumatic stress syndrome), mild cognitive impairment, ALS, Huntington's chorea, amnesia, B1 deficiency, schizophrenia, depression and bipolar disorder, stroke, hydrocephalus, subarachnoid hemorrhage, vascular insufficiency, brain tumor, epilepsy, Parkinson's disease, cerebral microangiopathy (Meyer, R. C., et al. Ann N Y Acad Sci 854, 307-17 (1998); Barrett, A. M. Postgrad Med 117, 47-53 (2005); Petersen, R. C. J Intern Med 256, 183-94 (2004); Calkins, M. E., et al. Am J Psychiatry 162, 1963-6 (2005)), pain medication, chemotherapy (“chemobrain”), oxygen deprivation, e.g, caused by a heart-lung machine, anesthesia, or near drowning, dementia (vascular, frontotemporal, Lewy-body, semantic, primary progressive aphasia, Pick's), progressive supranuclear palsy, corticobasal degeneration, Hashimoto encephalopathy, ADD, ADHD, dyslexia and other learning disabilities, Down syndrome, fragile X syndrome, Turner's syndrome, and fetal alcohol syndrome, for example. In addition to disease, progressive memory loss is a normal byproduct of the aging process.

The term MCI (“mild cognitive impairment”) is used to refer to a transitional zone between normal cognitive function and the development of clinically probable AD (Winblad, B. et al. J Intern Med 256, 240-6 (2004)). A variety of criteria have been utilized to define MCI, however they essentially have two major themes: (1) MCI refers to non-demented patients with some form of measurable cognitive defects and (2) these patients represent a clinical syndrome with a high risk of progressing to clinical dementia.

The phrase “improving learning and/or memory” refers to an improvement or enhancement of at least one parameter that indicates learning and memory. Improvement or enhancement is change of a parameter by at least 10%, optionally at least about 20%, at least about 30%, at least about 40%, at least about 50%, at least about 60%, at least about 70%, at least about 80%, at least about 90%, at least about 100%, at least about 150%, at least about 200%, etc. The improvement of learning and memory can be measured by any methods known in the art. For example, compounds described herein that improve learning and memory can be screened using Morris water maze (see, e.g., materials and methods section). See, also, Gozes et al., Proc. Natl. Acad. Sci. USA 93:427-432 (1996). Memory and learning can also be screened using any of the methods described herein or other methods that are well known to those of skill in the art, e.g., the Randt Memory Test, the Wechler Memory Scale, the Forward Digit Span test, or the California Verbal Learning Test.

The term “spatial learning” refers to learning about one's environment and requires knowledge of what objects are where. It also relates to learning about and using information about relationships between multiple cues in environment. Spatial learning in animals can be tested by allowing animals to learn locations of rewards and to use spatial cues for remembering the locations. For example, spatial learning can be tested using a radial arm maze (i.e., learning which arm has food) a Morris water maze (i.e., learning where the platform is). To perform these tasks, animals use cues from test room (positions of objects, odors, etc.). In human, spatial learning can also be tested. For example, a subject can be asked to draw a picture, and then the picture is taken away. The subject is then asked to draw the same picture from memory. The latter picture drawn by the subject reflects a degree of spatial learning in the subject.

Neuroplasticity is the lifelong ability of the brain to reorganize neural pathways based on new experiences. As we learn, we acquire new knowledge and skills through instruction or experience. In order to learn or memorize a fact or skill, there must be persistent functional changes in the brain that represent the new knowledge. These functional changes include neurite growth, synaptic plasticity, and neuroprotection, for example, including changes in neurons, glia, and vascular cells. See, e.g., Kandel, E. R., Schwartz, J. H., and Jessell, T. M. (2001). Principles of Neural Science. (4th ed.), New York: McGraw-Hill.

Learning disabilities is a general term that refers to a heterogeneous group of disorders manifested by significant difficulties in the acquisition and use of listening, speaking, reading, writing, reasoning, or mathematical abilities. Learning disabilities include ADD, ADHD, dyslexia, dysgraphia, dyscalcula, dyspraxia, and information processing disorders.

“Nitric oxide enhancing agents” include PDE5 inhibitors, such as Sildenafil (VIAGRA®, see, e.g., U.S. Pat. Nos. 5,250,534; 6,469,012), Tadalafil (CIALIS(®, see, e.g., U.S. Pat. Nos. 5,859,006; 6,140,329; 6,821,975; 6,943,166; 7,182,958); and Vardenafil (LEVITRA®, see, e.g., U.S. Pat. No. 6,362,178); nitric oxide donor molecules such as sodium nitroprusside (see, e.g., U.S. Pat. No. 6,358,536), amyl nitrate (see, e.g., U.S. Pat. No. 5,869,539), and nitroglycerin (see, e.g., U.S. Pat. No. 6,538,033); and statins, which are HMG Co A Reductase inhibitors, such as Atorvastatin (CADVET®, see, e.g., U.S. Pat. Nos. 4,572,909; 4,681,893; 5,273,995; 5,686,104; 5,969,156; 6,126,971; 6,455,574), Simvastatin (ZOCOS®, VYTORIN® (Ezetimbe and Simvastatin), see, e.g., U.S. Pat. Nos. 5,846,966; 7,229,982; RE37721), Lovastatin (ALTOPREV®, see, e.g., U.S. Pat. Nos. 5,916,595; 6,080,778; 6,485,748), Fluvastatin (LESCOL®, see, e.g., U.S. Pat. Nos. 5,354,772; 5,356,896), Pravastatin (PRAVACHOL®, see, e.g., U.S. Pat. Nos. 5,030,447; 5,180,589; 5,622,985), Mevastatin (see, e.g., U.S. Pat. No. 4,866,090), Pitavastatin (see, e.g., U.S. Pat. No. 7,208,623), and Rosuvastatin (CRESTOR®, see, e.g., U.S. Pat. No. 6,316,460; RE373314). Fasudil is known to stablize nitric oxide synthase messenger RNA (as do statins) the combined use of Fasudil and variants there of with nitric oxide enhancing agents further enhances pro-cognitive effects.

As used herein, “administering” refers to oral administration, administration as a suppository, topical contact, parenteral, intravenous, intraperitoneal, intramuscular, intralesional, oral, intranasal or subcutaneous administration, intrathecal administration, or the implantation of a slow-release device e.g., a mini-osmotic pump, to the subject.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

“Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid.

“Unnatural amino acids” are not encoded by the genetic code and can, but do not necessarily have the same basic structure as a naturally occurring amino acid. Unnatural amino acids include, but are not limited to azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, omithine, pentylglycine, pipecolic acid and thioproline.

“Amino acid mimetics” refers to chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid (i.e., hydrophobic, hydrophilic, positively charged, neutral, negatively charged). Exemplified hydrophobic amino acids include valine, leucine, isoleucine, methionine, phenylalanine, and tryptophan. Exemplified aromatic amino acids include phenylalanine, tyrosine and tryptophan. Exemplified aliphatic amino acids include serine and threonine. Exemplified basic aminoacids include lysine, arginine and histidine. Exemplified amino acids with carboxylate side-chains include aspartate and glutamate. Exemplified amino acids with carboxamide side chains include asparagines and glutamine. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “alkyl” refers to a straight or branched, saturated, aliphatic radical having the number of carbon atoms indicated. For example, C₁-C₆ alkyl includes, but is not limited to, methyl, ethyl, propyl, butyl, pentyl, hexyl, iso-propyl, iso-butyl, sec-butyl, tert-butyl, etc.

As used herein, the term “halogen” refers to fluorine, chlorine, bromine and iodine.

As used herein, the term “heterocycle” refers to a ring system having from 5 to 8 ring members and 2 nitrogen heteroatoms. For example, heterocycles useful in the present invention include, but are not limited to, pyrazolidine, imidazolidine, piperazine and homopiperazine. The heterocycles of the present invention are N-linked, meaning linked via one of the ring heteroatoms.

As used herein, the term “hydrate” refers to a compound that is complexed to at least one water molecule. The compounds of the present invention can be complexed with from 1 to 10 water molecules.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “prodrug” refers to compounds that when administered to the subject undergo a modification or transformation via in vivo processes that result in formation of the desired drug. Accordingly, the prodrug is administered, is transformed into the drug, and the drug then performs the desired function. Prodrugs can be prepared by techniques known to one skilled in the art.

As used herein, the term “salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of the present invention are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

As used herein, the term “subject” refers to animals such as mammals, including, but not limited to, primates (e.g., humans), cows, sheep, goats, horses, dogs, cats, rabbits, rats, mice and the like. In certain embodiments, the subject is a human.

As used herein, the terms “therapeutically effective amount” or “therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

II. Method for Improving Memory and Learning

The present invention provides methods for improving memory and learning by the administration of a compound Formula I, Formula II or Formula III, or a combination thereof, or another compound shown in FIG. 10. As shown in the examples below, the compounds of the present invention enhance memory and learning. For example, aging rats receiving hydroxyfausidil perform at a level comparable to young rats. Furthermore, as described in Papassotiropoulos et al., Science 314:475-314 (2006), the KIBRA gene is associated with episodic memory. The SNP rs7070145 is a common T to C substitution in the ninth intron of KIBRA accession number NM_{—}015238). Carries of the T allele where shown to have 24% better free recall performance (5 minutes) and 19% better free recall performance (24 hours) in a verbal delayed recall test for episodic memory performance. The compounds can be administered orally, parenterally, or nasally, for example. For long term administration, lower doses can be used. The compounds can be used in combination with other drugs to treat disease states or improve learning and memory.

III. Compounds for Improving and Enhancing Memory and Learning

Compounds useful in the methods of the present invention include compounds of Formula I: 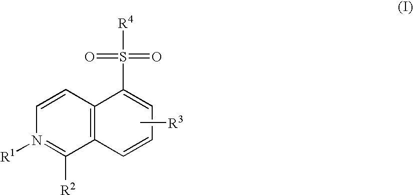
R¹ of Formula I, is absent or is hydrogen or C₁₋₆alkyl. R² is hydrogen, hydroxy or halogen. R³ is hydrogen or C₁₋₆alkyl. R⁴ is an N-linked heterocyclic ring system having from 5 to 8 ring members and two N ring heteroatoms, substituted with 0-3 R⁵ groups, wherein each R⁵ is hydrogen, C₁₋₆alkyl, benzyl or phenyl. The compounds of Formula I can also be in a salt, hydrate or solvate form, or a combination.

Compounds of Formula I, and their salts and hydrates, can be prepared using existing means (see U.S. Pat. Nos. 4,678,783 and 5,942,505, and European Patent No. 187,371).

In some embodiments, R⁴ is a 7-membered heterocyclic ring system. In other embodiments, the compound has Formula Ia:

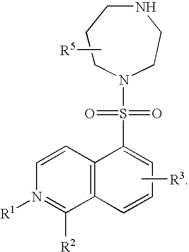

In other embodiments, the compound of Formula I is:

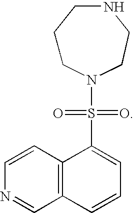

In still other embodiments, the compound of Formula I is:

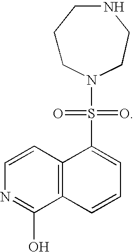

In another embodiment, the compound is the HCl salt. In some embodiments, the compound of Formula I is:

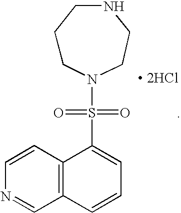

In a further embodiment, the compound is the hydrate. In another embodiment, the compound of Formula I is: 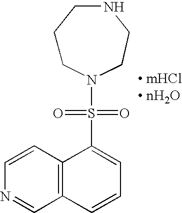
wherein m is 1 or 2 and n is from ½ to 3.

Compounds useful in the methods of the present invention include compounds of Formula II: 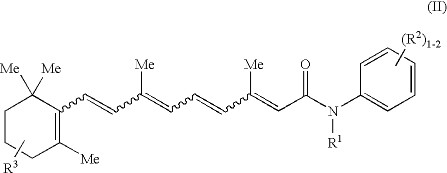
wherein R¹ is hydrogen or C₁₋₆ alkyl. Each R² is hydrogen, C₁₋₆ alkyl, hydroxy or —O—C₁₋₆ alkyl. R³ is hydrogen or C₁₋₆ alkyl. Each represents that the double bond to which it is attached is cis or trans. The compounds of Formula II can also be in a salt, hydrate or solvate form, or a combination.

In some embodiments, the compound is of Formula IIa:

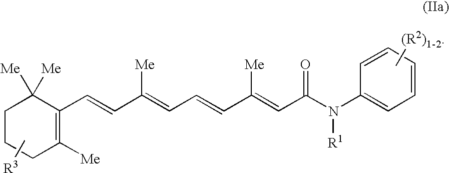

In other embodiments, the compound of Formula II is:

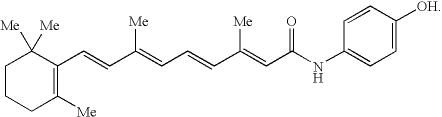

Additional compounds useful in the methods of the present invention include retinamides and retinamide analogs.

Compounds useful in the methods of the present invention also include compounds of Formula III:
(R¹)ₓ—C—B-Tyr-Arg-Arg-A-A-Arg-Arg-Trp-Arg-Lys-B—(R²)_{y}
and conservatively modified variations thereof. R¹ is an amino acid sequence comprising from 1 to about 40 amino acids wherein each amino acid is a naturally occurring amino acid or an amino acid analog. R² is an amino acid sequence comprising from 1 to about 40 amino acids wherein each amino acid is a naturally occurring amino acid or an amino acid analog. A represents glycine or alanine; B represents isoleucine, leucine, methionine or valine; C represents serine or threonine; and x and y are independently selected and are equal to zero or one.

In some embodiments, the compound of Formula III is:
(R¹)ₓ-Ser-Ile-Tyr-Arg-Arg-Gly-Ala-Arg-Arg-Trp-Arg-Lys-Leu —(R²)_{y}
and conservatively modified variations thereof. R¹ is an amino acid sequence comprising from 1 to about 40 amino acids wherein each amino acid is a naturally occurring amino acid or an amino acid analog. R² is an amino acid sequence comprising from 1 to about 40 amino acids wherein each amino acid is a naturally occurring amino acid or an amino acid analog. A represents glycine or alanine; B represents isoleucine, leucine, methionine or valine; C represents serine or threonine; and x and y are independently selected and are equal to zero or one.

In another embodiment, the compound of Formula III is:
(R¹)ₓ-Ser-Ile-Tyr-Arg-Arg-Gly-Ala-Arg-Arg-Trp-Arg-Lys-Leu —(R²)_{y}
and conservatively modified variations thereof, in which x and y are zero.
IV. Formulations for Improving Memory and Learning

The compounds of the present invention can be formulated in a variety of different manners known to one of skill in the art. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington 's Pharmaceutical Sciences, 20^{th} ed., 2003, supra). Effective formulations include oral and nasal formulations, formulations for parenteral administration, and compositions formulated for with extended release.

Formulations suitable for oral administration can consist of (a) liquid solutions, such as an effective amount of a compound of the present invention suspended in diluents, such as water, saline or PEG 400; (b) capsules, sachets, depots or tablets, each containing a predetermined amount of the active ingredient, as liquids, solids, granules or gelatin; (c) suspensions in an appropriate liquid; (d) suitable emulsions; and (e) patches. The pharmaceutical forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents. Preferred pharmaceutical preparations can deliver the compounds of the invention in a sustained release formulation.

Pharmaceutical preparations useful in the present invention also include extended-release formulations. In some embodiments, extended-release formulations useful in the present invention are described in U.S. Pat. No. 6,699,508, which can be prepared according to U.S. Pat. No. 7,125,567, both patents incorporated herein by reference.

The pharmaceutical preparations are typically delivered to a mammal, including humans and non-human mammals. Non-human mammals treated using the present methods include domesticated animals (i.e., canine, feline, murine, rodentia, and lagomorpha) and agricultural animals (bovine, equine, ovine, porcine).

In practicing the methods of the present invention, the pharmaceutical compositions can be used alone, or in combination with other therapeutic or diagnostic agents.

V. Administration for Improving Memory and Learning

The compounds of the present invention can be administered as frequently as necessary, including hourly, daily, weekly or monthly. The compounds utilized in the pharmaceutical method of the invention are administered at the initial dosage of about 0.0001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the type and stage of disease diagnosed in a particular patient. The dose administered to a patient, in the context of the present invention should be sufficient to effect a beneficial therapeutic response in the patient over time. The size of the dose also will be determined by the existence, nature, and extent of any adverse side-effects that accompany the administration of a particular compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired. Doses can be given daily, or on alternate days, as determined by the treating physician. Doses can also be given on a regular or continuous basis over longer periods of time (weeks, months or years), such as through the use of a subdermal capsule, sachet or depot, or via a patch.

The pharmaceutical compositions can be administered to the patient in a variety of ways, including topically, parenterally, intravenously, intradermally, subcutaneously, intramuscularly, colonically, rectally or intraperitoneally. Preferably, the pharmaceutical compositions are administered parenterally, topically, intravenously, intramuscularly, subcutaneously, orally, or nasally, such as via inhalation.

In practicing the methods of the present invention, the pharmaceutical compositions can be used alone, or in combination with other therapeutic or diagnostic agents. The additional drugs used in the combination protocols of the present invention can be administered separately or one or more of the drugs used in the combination protocols can be administered together, such as in an admixture. Where one or more drugs are administered separately, the timing and schedule of administration of each drug can vary. The other therapeutic or diagnostic agents can be administered at the same time as the compounds of the present invention, separately or at different times.

VI. Predictive and Diagnostic Methods

The present invention provides methods of predicting and/or diagnosing Alzheimer's disease by detecting the presence or absence of the C-alelle for KIBRA SNP rs17070145. As used herein, the term “diagnosis” refers to distinguishing aiding in a diagnosis of MCI or Alzheimer's disease.

Nucleic acid binding molecules such as probes, oligonucleotides, oligonucleotide arrays, and primers can be used in assays to detect the marker in patient samples, e.g., RT-PCR. In one embodiment, RT-PCR is used according to standard methods known in the art. In another embodiment, PCR assays such as Taqman® assays available from, e.g., Applied Biosystems, can be used to detect nucleic acids and variants thereof. In other embodiments, qPCR and nucleic acid microarrays can be used to detect nucleic acids. Reagents that bind to selected biomarkers can be prepared according to methods known to those of skill in the art or purchased commercially.

Analysis of nucleic acids can be achieved using routine techniques such as Southern analysis, reverse-transcriptase polymerase chain reaction (RT-PCR), or any other methods based on hybridization to a nucleic acid sequence that is complementary to a portion of the marker coding sequence (e.g., slot blot hybridization) are also within the scope of the present invention. Applicable PCR amplification techniques are described in, e.g., Ausubel et al. and Innis et al., supra. General nucleic acid hybridization methods are described in Anderson, “Nucleic Acid Hybridization,” BIOS Scientific Publishers, 1999. Amplification or hybridization of a plurality of nucleic acid sequences (e.g., genomic DNA, mRNA or cDNA) can also be performed from MRNA or cDNA sequences arranged in a microarray. Microarray methods are generally described in Hardiman, “Microarrays Methods and Applications: Nuts & Bolts,” DNA Press, 2003; and Baldi et al., “DNA Microarrays and Gene Expression: From Experiments to Data Analysis and Modeling,” Cambridge University Press, 2002.

Analysis of nucleic acid markers and their variants can be performed using techniques known in the art including, without limitation, microarrays, polymerase chain reaction (PCR)-based analysis, sequence analysis, and electrophoretic analysis. A non-limiting example of a PCR-based analysis includes a Taqman® allelic discrimination assay available from Applied Biosystems. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nat. Biotechnol., 16:381-384 (1998)), and sequencing by hybridization. Chee et al., Science, 274:610-614 (1996); Drmanac et al., Science, 260:1649-1652 (1993); Drmanac et al., Nat. Biotechnol., 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Other methods for detecting nucleic acid variants include, e.g., the INVADER® assay from Third Wave Technologies, Inc., restriction fragment length polymorphism (RFLP) analysis, allele-specific oligonucleotide hybridization, a heteroduplex mobility assay, single strand conformational polymorphism (SSCP) analysis, single-nucleotide primer extension (SNUPE) and pyrosequencing.

Antibody reagents can be used in assays to detect expression levels of the marker of the invention (polypeptide variant encoded by the C- or T-variant of the rs17070145 KIBRA SNP) in patient samples using any of a number of immunoassays known to those skilled in the art. immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, C A; Kit #449430) and can be performed using a Behring Nephelometer Analyzer (Fink et al., J. Clin. Chem. Clin. Biochem., 27:261-276 (1989)).

Specific immunological binding of the antibody to nucleic acids can be detected directly or indirectly. Direct labels include fluorescent or luminescent tags, metals, dyes, radionuclides, and the like, attached to the antibody. An antibody labeled with iodine-125 (¹²⁵I can be used. A chemiluminescence assay using a chemiluminescent antibody specific for the nucleic acid is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissarnine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

Useful physical formats comprise surfaces having a plurality of discrete, addressable locations for the detection of a plurality of different markers. Such formats include microarrays and certain capillary devices. See, e.g., Ng et al., J. Cell Mol. Med., 6:329-340 (2002); U.S. Pat. No. 6,019,944. In these embodiments, each discrete surface location may comprise antibodies to immobilize one or more markers for detection at each location. Surfaces may alternatively comprise one or more discrete particles (e.g., microparticles or nanoparticles) immobilized at discrete locations of a surface, where the microparticles comprise antibodies to immobilize one or more markers for detection.

Analysis can be carried out in a variety of physical formats. For example, the use of microtiter plates or automation could be used to facilitate the processing of large numbers of test samples. Alternatively, single sample formats could be developed to facilitate diagnosis or prognosis in a timely fashion.

Alternatively, the antibodies or nucleic acid probes of the invention can be applied to sections of patient biopsies immobilized on microscope slides. The resulting antibody staining or in situ hybridization pattern can be visualized using any one of a variety of light or fluorescent microscopic methods known in the art.

In another format, the various markers of the invention also provide reagents for in vivo imaging such as, for instance, the imaging of labeled regents that detect the nucleic acids or encoded proteins of the biomarkers of the invention. For in vivo imaging purposes, reagents that detect the presence of proteins encoded by cancer biomarkers, such as antibodies, may be labeled using an appropriate marker, such as a fluorescent marker.

VII. Compositions, Kits and Integrated Systems

The invention provides compositions, kits and integrated systems for practicing the assays described herein using antibodies specific for the polypeptides or nucleic acids specific for the polynucleotides of the invention.

Kits for carrying out the diagnostic assays of the invention typically include a probe that comprises an antibody or nucleic acid sequence that specifically binds to polypeptides or polynucleotides of the invention, and a label for detecting the presence of the probe. The kits may include several antibodies or polynucleotide sequences encoding polypeptides of the invention, e.g., a cocktail of antibodies that recognize the proteins encoded by the biomarkers of the invention.

### VI. EXAMPLES

### Example 1

### Improvement in Memory by Treatment with Hydroxvfasudil

Hydroxyfasudil was tested in vivo by administering to aged rats (18 months old) two doses of 0.75 mg/kg and 0.375 mg/kg, delivered subcutaneously each day for a period of 4-5 days before testing. During testing, hydroxyfasudil was administered in the morning of each testing day. Two control groups were used, with one group being untreated and the other group treated with the vehicle (saline). Both control groups include 9 members, with an age of 4 months.

The rats were then tested using the water escape radial arm maze test. The radial arm maze is a swim-based test without the need for food deprivation. The maze consists of 8 radial arms, with 4 of the arms having a submerged platform. The rat was placed in the start arm, which remains constant throughout the test and does not contain a platform. The rat was given 180 seconds to locate a platform. If the rat located a platform, the rat was allowed 15 seconds on the platform, and was then removed. When a rat found a platform, that platform was removed prior to beginning the next trial. Each rat was subjected to 4 trials per day, with 1 trial per platform and 30 seconds between trials. Each rat was tested over a period of 12 days, with the first day consisting of training, days 2-7 considered “initial,” and days 8-12 considered “latter.”

The rats were also tested using the Morris water maze. The Morris water maze consists of a circular water bath with a single platform. The bath is divided into 4 quadrants and various annuli. A rat was placed in the water bath and the time and distance that the rat required to reach the platform was measured. The solving strategy used by the rat was also noted. The movement of the rat was tracked using a digital tracking system. Each rat underwent 4 trials per day, with entry into the maze varied by quadrant for each trial. The Morris water maze included 5 days of testing, with the first 4 days involving testing and day 5 involving a probe trial.

After testing, the rats were terminated and the brain dissected.

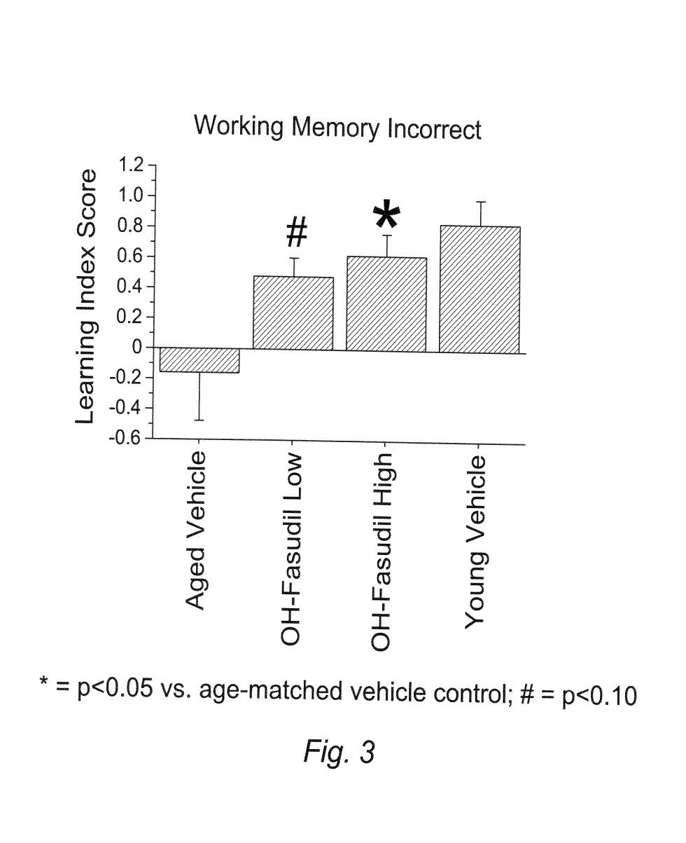

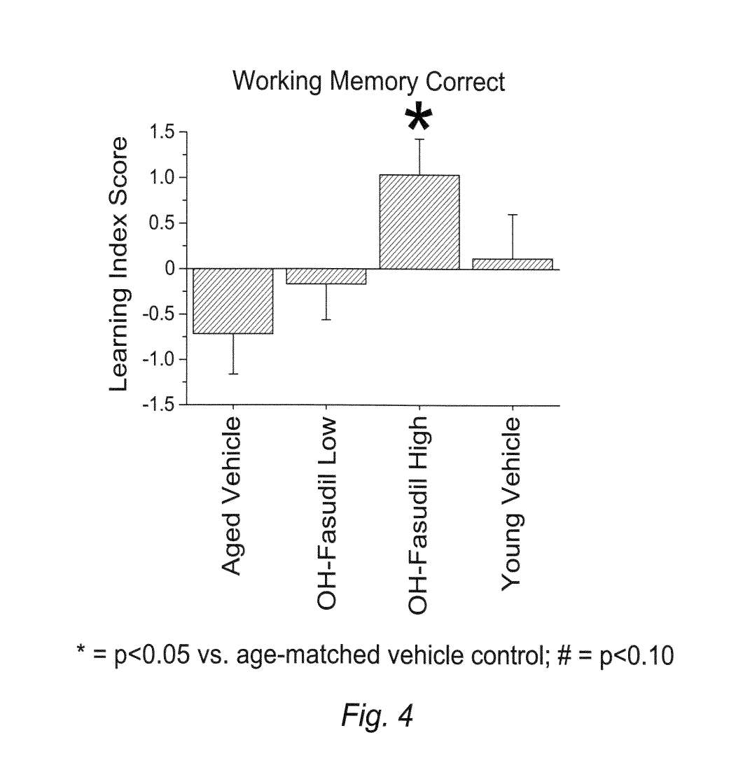

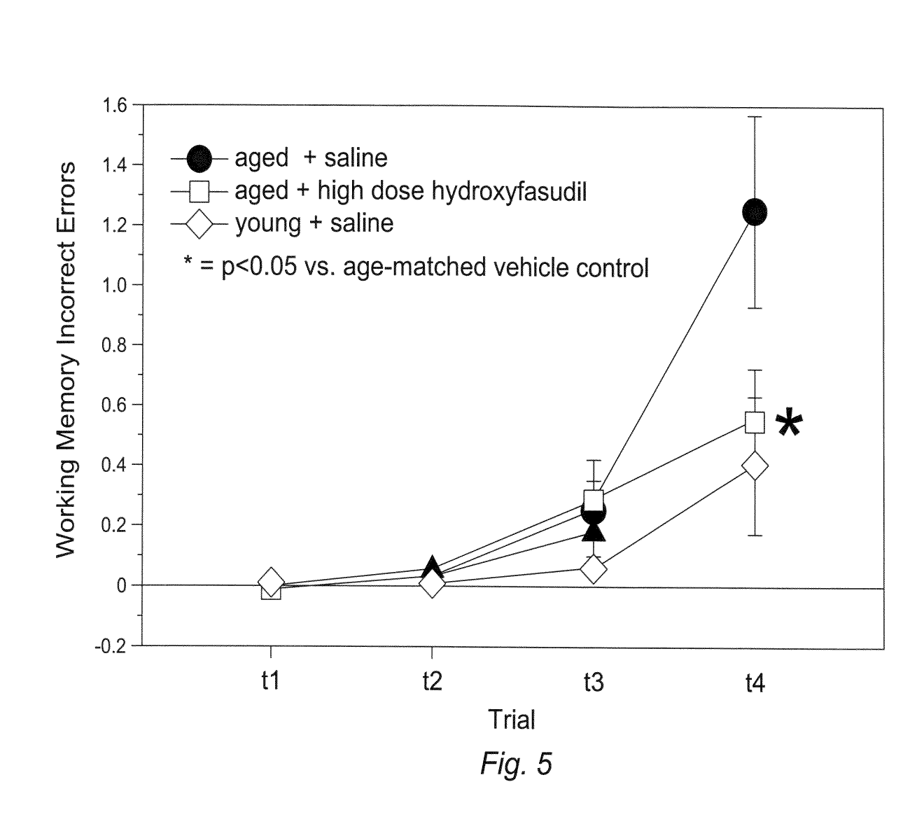

FIGS. 3-4 show improved learning scores (orthogonal measures of working memory) with administration of hydroxyfasudil, with higher doses of hydroxyfasudil achieving greater improvements of learning versus smaller does. However, smaller doses could be useful for long term administration. FIGS. 3-5 also show improved memory with administration of hydroxyfasudil, with higher doses of hydroxyfasudil achieving greater improvements of memory versus smaller doses of hydroxyfasudil. However, smaller doses could be useful for long term administration.

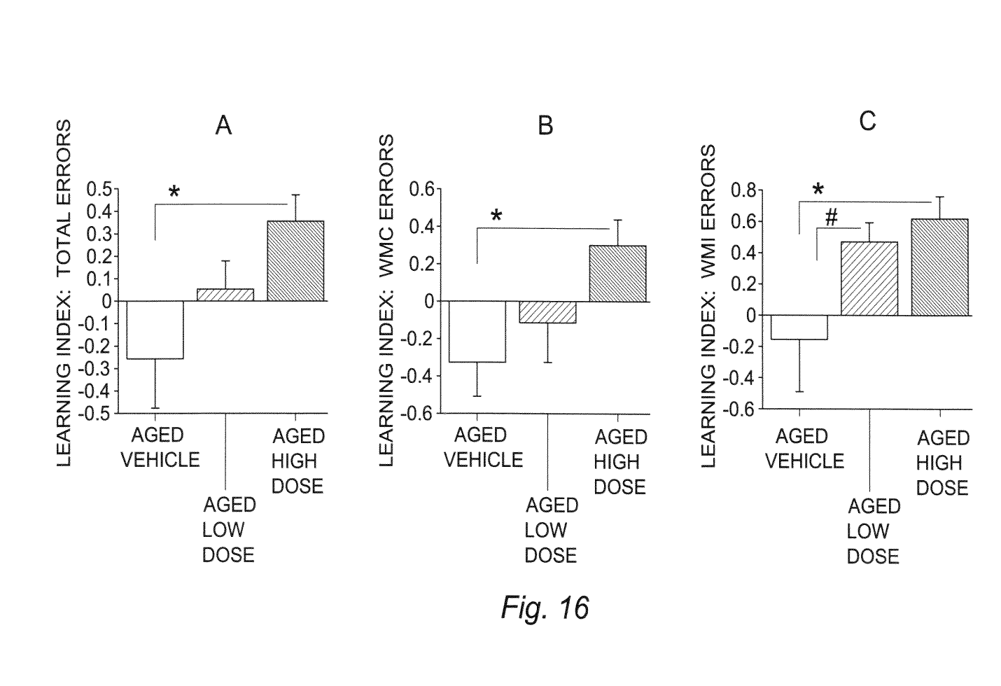

Additional efficacy testing was performed using twenty-seven 18-month old male rats were utilized and divided randomly into three treatment groups: saline vehicle (“Aged Vehicle”), hydroxyfasudil in saline at a dose of 0.1875 mg per day (“Aged Low Dose”), and hydroxyfasudil at a dose of 0.3750 mg per day (“Aged High Dose”). Daily injections of the assigned substrate began four days prior to behavioral testing and continued throughout testing, with injections given approximately one hour before daily testing ensued. Animals were tested 4 trials per day for 12 consecutive days. Following the water radial arm maze, spatial reference memory was assessed using the Morris water maze. This testing consisted of 4 trials per day for 4 days with an additional probe trial on the final day. The data in FIG. 16 demonstrates improved memory with administration of hydroxyfasudil.

### Example 2

### Improvement in Memory by Treatment with Fenretinide

Using the protocol described above for hydroxyfasudil, rats were treated with from 1 mg/kg to 2 mg/kg of fenretinide. The rats were subjected to the Radial Arm Maze and the Morris Water Maze described above.

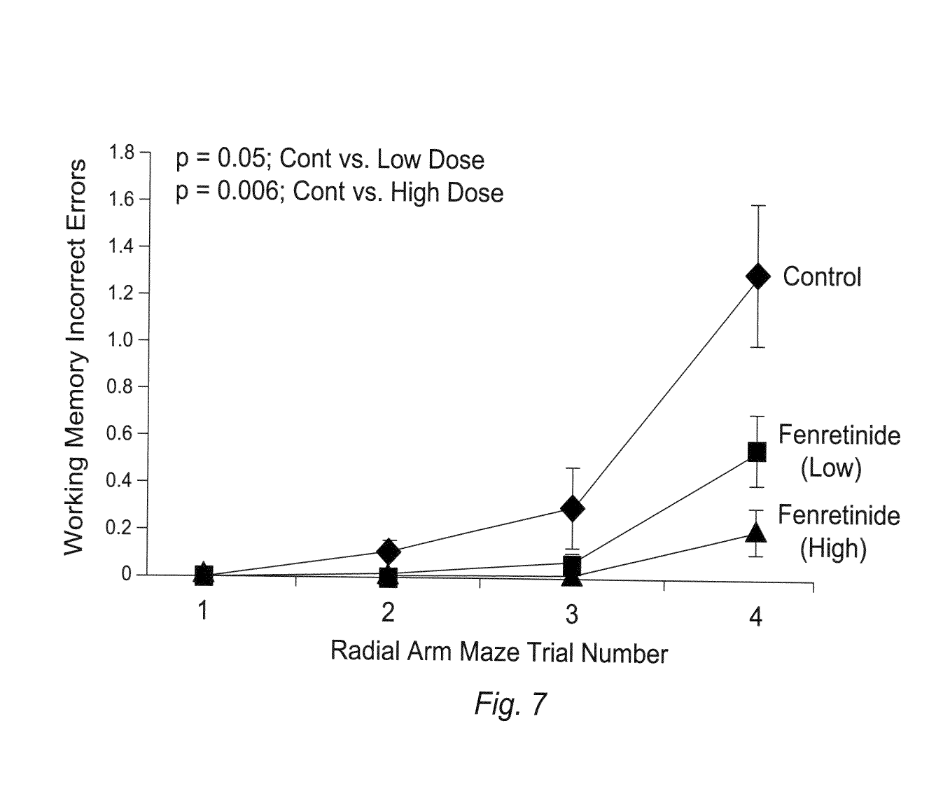

FIGS. 7 shows that treatment with fenretinide lowered the number of incorrect errors made by the rats, versus the control. In addition, FIG. 7 demonstrates that treatment with greater doses of fenretinide improved memory more than smaller doses of fenretinide.

### Example 3

### Improvement in Memory by Treatment with Fasudil

Aged animals were pre-loaded for 14 days at 10mg/kg/day (FAS LOW DOSE+PRIMING) or at 3 mg/kg/day (FAS LOW DOSE). After 14 days all animals were administered 3 mg/kg/day. Vehicle was normal saline (0.9%). The rats were subjected to the Radial Arm Maze and the Morris Water Maze described above.

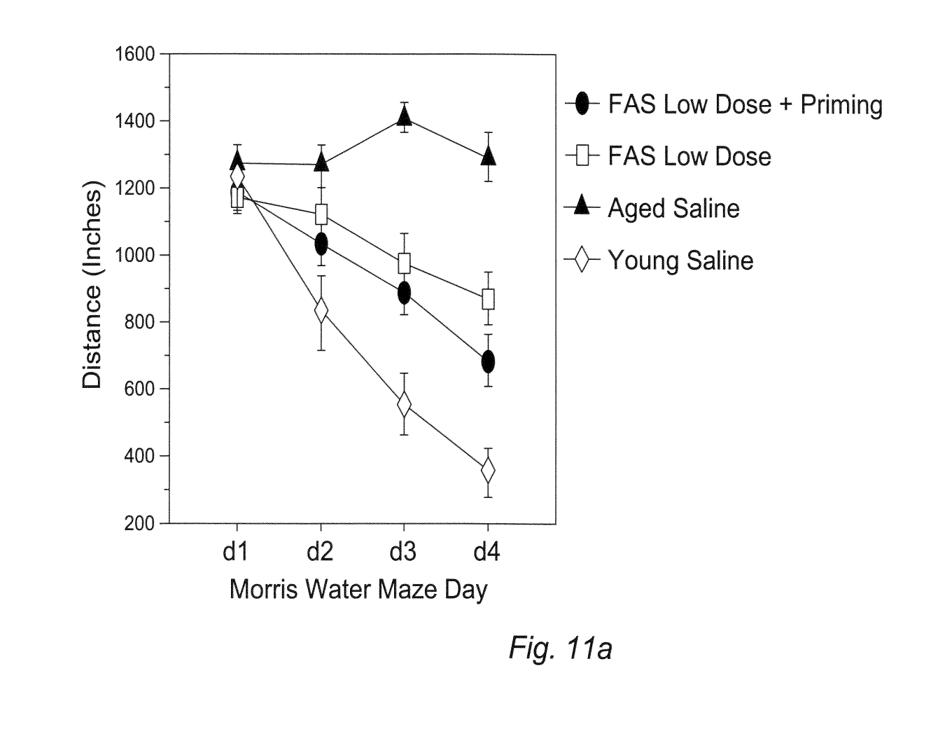

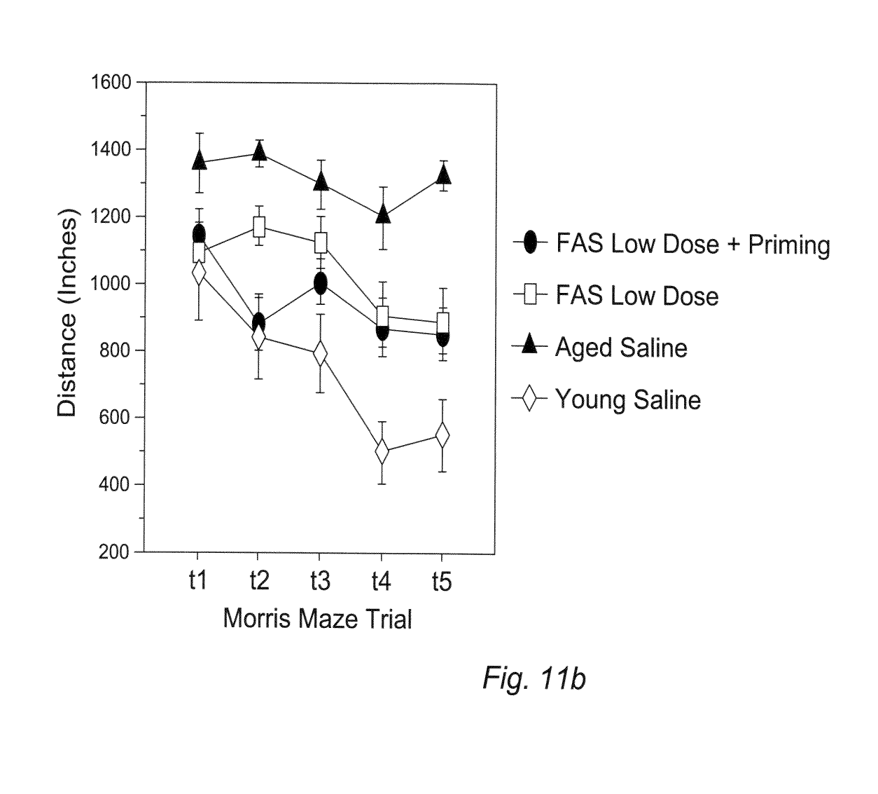

FIGS. 11A-B show improved reference memory performance as the number of days taking the test increases (FIG. 11A) and the number of trials increases (FIG. 11B).

Fasudil was also delivered via daily sub-cutaneous injections to aged (18-months old) rats at doses of 3 mg/kg/day (n=8) or 30 mg/kg/day (n=8). Controls included both aged and young (4-months old) rats injected daily with vehicle (normal saline). The low dose for Fasudil was based upon the previous study showing efficacy with 0.750 mg/kg/day hydroxyfasudil and the high dose was based on that reported in the literature as a commonly used therapeutic dose.

### Example 4

### Improvement in Memory by Treatment with Fasudil II

This example provides additional evidence of improvement in memory following treatment with fasudil.

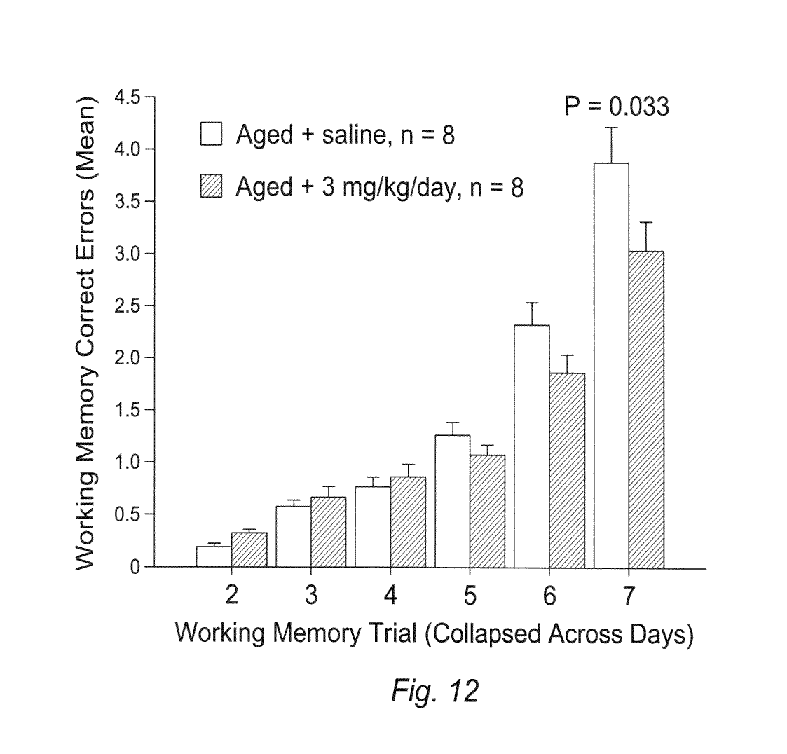

This experiment included a decreased pre-loading phase, a more difficult version of the working memory trial (where 7 out of 8 arms were platformed), and an elongated version of the Morris maze (7 days plus a “probe” trial). Fasudil was delivered via daily sub-cutaneous injections to aged (18-months old) rats at doses of 3 mg/kg/day (n=8) or 10 mg/kg/day (n=8). Controls included both aged and young (4-months old) rats injected daily with vehicle (normal saline). The low dose for Fasudil was based upon the evidence in Example 3 showing efficacy and the high dose was based on that reported in the literature as a commonly used low therapeutic dose. There was a one day loading period for this experiment. After 7 days of drug delivery, the lOmg/kg/day animals began to show similar trauma as in the previous study and were switched to the low dose. A significant improvement in working memory performance was observed, especially in trails 6 and 7 of the water escape radial arm maze, the most demanding trials with regards to working memory “load”. See FIG. 12.

### Example 5

### Correlation of KIBRA SNP Associated with Normal Episodic Memory Performance with Genetic Risk Factor for Alzheimer's Disease

This example provides evidence that the KIBRA SNP associated with normal episodic memory performance is also a genetic risk factor for development of Alzheimer's disease.

A sampling of 26 single nucleotide polymorphisms spanning the KIDRA locus were examined in a post-mortem confirmed (i.e. positive for amyloid plaques and neurofibriallary tangles) and clinically demented at the time of death cohort of Alzheimer's patients (n=595) or matched healthy control donors (n=320). The most significant haplotype identified in this analysis included SNP rs17070145, the KIBRA SNP linked to episodic memory performance. The allele associated with Alzheimer's was the C-allele, the allele associated with poorer memory performance. Based on this initial finding, rs17070145 was examined in a large cohort of ante-mortem diagnosed patients (n=1,373) and matched controls (n=785) from the United States, Norway, Netherlands, and Germany. The Cochran-Mantel-Haenszel test for significance yielded p=0.0013 for the C-allele of rs17070145. This large collection of genotyping data indicates that the same KIBRA SNP and haplotype associated with normal episodic memory performance is also a genetic risk factor for Alzheimer's disease.

### Example 6

### Transcriptional Regulation of MIBRA and PKC-zeta

This example provides a description of the transcriptional regulation of KIBRA and its demonstrated kinase, PKC-zeta, in brain regions of patients with mild cognitive impairment (MCI), Alzheimer's Disease, and matched cognitively normal controls.

Two brain regions known to be involved in both memory and AD progression were investigated, the hippocampus and the middle temporal gyrus (MTG). In the context of MCI and within the hippocampus, PKC-zeta message was decreased over 5-fold (p=7.6E-05) while KIBRA message was not significantly changed. In the hippocampus of AD patients, PKC-zeta was downregulated 2.67-fold (p=2.2E-06) and KIBRA message was also increased over 3-fold (p=2.7E-05). Within the middle temporal gyrus in MCI patients, both KIBRA (−1.61-fold, p=2.3E-04) and PKC-zeta (−2.52-fold, p=4.7E-04) messages were decreased. While in the MTG in AD patients, PKC-zeta message was also significantly decreased over 3-fold (p=1.0E-04) while KIBRA was increased 2.69-fold (p=3.3E-04). Note that this data is further illustrated in the table below and that all of these p-values have been corrected for multiple testing comparisons.

| | | | | FOLD |
|---|---|---|---|---|
| BRAIN REGION | | DISORDER | P-VALUE | -CHANGE |
| HIPPOCAMPUS | KIBRA | AD | 2.7E−05 | 3.08 |
| | PRKCZ | AD | 2.2E−06 | −2.67 |
| | KIBRA | MCI | N/S | N/S |
| | PRKCZ | MCI | 7.6E−05 | −5.19 |
| MIDDLE | KIBRA | AD | 3.3E−04 | 2.69 |
| TEMPORAL | PRKCZ | AD | 1.0E−04 | −3.24 |
| GYRUS | [symbol] | [symbol] | [symbol] | [symbol] |
| | [symbol] | [symbol] | [symbol] | [symbol] |

### Example 7

### Validation of the Memory Pathway

This example provides a validation of the memory pathway.

Three hundred and fifty one young adults (median age: 22 years, range: 18-48 years) from Switzerland were recruited. Genetic association studies in outbred populations such as the present one may be prone to false-positivity because non-random genetic heterogeneity within the study sample (population structure) can lead to spurious associations between a genetic marker and a phenotype (Freedman, M. L. et al., Nat. Genet. 36, 388 (2004)). A structured association analysis (Pritchard, J. K. & Rosenberg, N. A. Am. J. Hum. Genet. 65, 220 (1999)) was performed. It was discovered that the allele-frequency divergence in this population was moderate and that the participants' genetic backgrounds formed one normally distributed cluster (P=0.6). Ten subjects were identified as outliers (probability of cluster allocation lower than 25%) and were therefore excluded from the genetic association studies.

The remaining population (n=341) was stratified into 4 groups according to their performance in a verbal memory task which quantified the retrieval success 5 min after learning of a word list comprising 30 semantically unrelated nouns. Each of these quartiles were genotyped at 502,627 SNPs. Poor performing SNPs were discarded, and both single-point and sliding window (multi-point) statistical approaches were employed to select SNPs associated with performance at high statistical confidence. Two SNPs were significant with both analysis strategies: rs17070145 and rs6439886. Both SNPs map within genes expressed in the human brain: rs17070145 is a common T→C substitution within the ninth intron of KIBRA (NM_{—}015238), encoding a neuronal protein, and rs6439886 is a common T→C substitution within the first intron of CLSTN2 (encoding the synaptic protein calsyntenin 2) (NM_{—}022131).

Individual genotyping in the original Swiss cohort (n=341) validated the whole-genome array-based scanning results and revealed that both the KIBRA and CLSTN2 SNPs were significantly associated with differential human memory performance (see, e.g., Carriers of the rs17070145*T allele had 24% better free recall performance 5 minutes after word presentation (P=0.000004) and 19% better free recall performance 24 hours after word presentation (P=0.0008) than non-carriers. SNP rs6439886 yielded similar results with lower effect sizes (see Table 1, see also Papassotiropoulos et al., Science 314:475-478 (2006); see also PCT/IUS07/61112). Both the 5-min and the 24-h delayed free recall reflect episodic, hippocampus-dependent memory (Squire, L. R. & Alvarez, P. Curr. Opin. Neurobiol. 5, 169 (1995)). Neither SNP was associated with performance on immediate recall tests, indicating that the allele-dependent differences in delayed episodic memory were not caused by allelic effects on confounding factors such as motivation, attention or working memory.

Both SNPs were further evaluated in a second, independent population of 256 cognitively normal older participants (median age: 55 years, range: 20-81 years) from the United States. The KIBRA SNP showed significant association with episodic memory with the same direction of effect: T allele carriers had significantly better memory scores than non-carriers in two different tests of episodic memory, the Rey Auditory Verbal Learning Test (AVLT) (Rosenberg, S.J. et al. J. Clin. Psychol. 40, 785 (1984)) and the Buschke's Selective Reminding Test (SRT) (Owen, E. H., et al. Neuroscience 80, 1087-99 (1997)) (Table 2, see also Papassotiropoulos et al., Science 314:475-478 (2006); see also PCT/US07/61112)). There were no allele-dependent differences in the outcome of the Wisconsin Card Sorting Test and on the Paced Auditory Serial Attention Task, suggesting that rs17070145 is not associated with executive functions, attention or working memory in this population. SNP rs6439886 failed to show significant association with episodic memory in this older population. Beside the possibility of false-positivity in the first sample for this particular SNP, the lack of significance in the second population may also be related to differences in ethnicity and mean age between the populations.

TABLE 1
Association of SNPs rs17070145 (KIBRA) and rs6439886
(CLSTN2) with verbal episodic memory in the Swiss population.

| | | Immediately | Words recalled | Words recalled |
|---|---|---|---|---|
| | | recalled words | after 5 min | after 24 h |
| | n | (mean ± SEM) | (mean ± SEM) | (mean ± SEM) |
| rs17070145* | | | | |
| CC | 164 | 23.6 ± 0.3 | 7.6 ± 0.2^{a} | 6.7 ± 0.2^{b} |
| CT/TT | 169 | 24.1 ± 0.3 | 9.4 ± 0.2^{a} | 8.0 ± 0.2^{b} |
| rs6439886 | | | | |
| TT | 265 | 23.9 ± 0.2 | 8.4 ± 0.2^{c} | 7.3 ± 0.2^{d} |
| TC/CC | 76 | 24.2 ± 0.4 | 9.8 ± 0.4^{c} | 8.4 ± 0.4^{d} |

Means with common superscripts are significantly different according to multifactorial analysis of variance.
*Genotype calls of 8 subjects failed to pass the quality control criteria.
^{a}P = 0.000004,
^{b}P = 0.0008,
^{c}P = 0.002,
^{d}P = 0.022

TABLE 2
Association of SNPs rs17070145 (KIBRA) and rs6439886 (CLSTN2) with
verbal episodic memory in the US population*.

| | | Immediately | Words recalled | Free recall |
|---|---|---|---|---|
| | | recalled | after | of words |
| | | words (AVLT) | 30 min (AVLT) | (SRT) |
| | n | (mean ± SEM) | (mean ± SEM) | (mean ± SEM) |
| rs17070145 | | | | |
| CC | 126 | 9.4 ± 0.3 | 8.5 ± 0.3^{a} | 83.7 ± 1.2^{b} |
| CT/TT | 130 | 10.0 ± 0.3 | 9.7 ± 0.3^{a} | 90.3 ± 1.1^{b} |
| rs6439886^{#} | | | | |
| TT | 185 | 9.7 ± 0.2 | 9.1 ± 0.2 | 88.4 ± 0.9 |
| TC/CC | 64 | 9.9 ± 0.4 | 9.2 ± 0.4 | 88.9 ± 1.6 |

Means with common superscripts are significantly different according to multifactorial analysis of variance.
*The SRT was completed by 200 participants (98 CC carriers and 102 CT & TT carriers of rs17070145)
^{#}Genotype calls of 7 subjects failed to pass the quality control criteria.
^{a}P = 0.004,
^{b}P = 0.00005

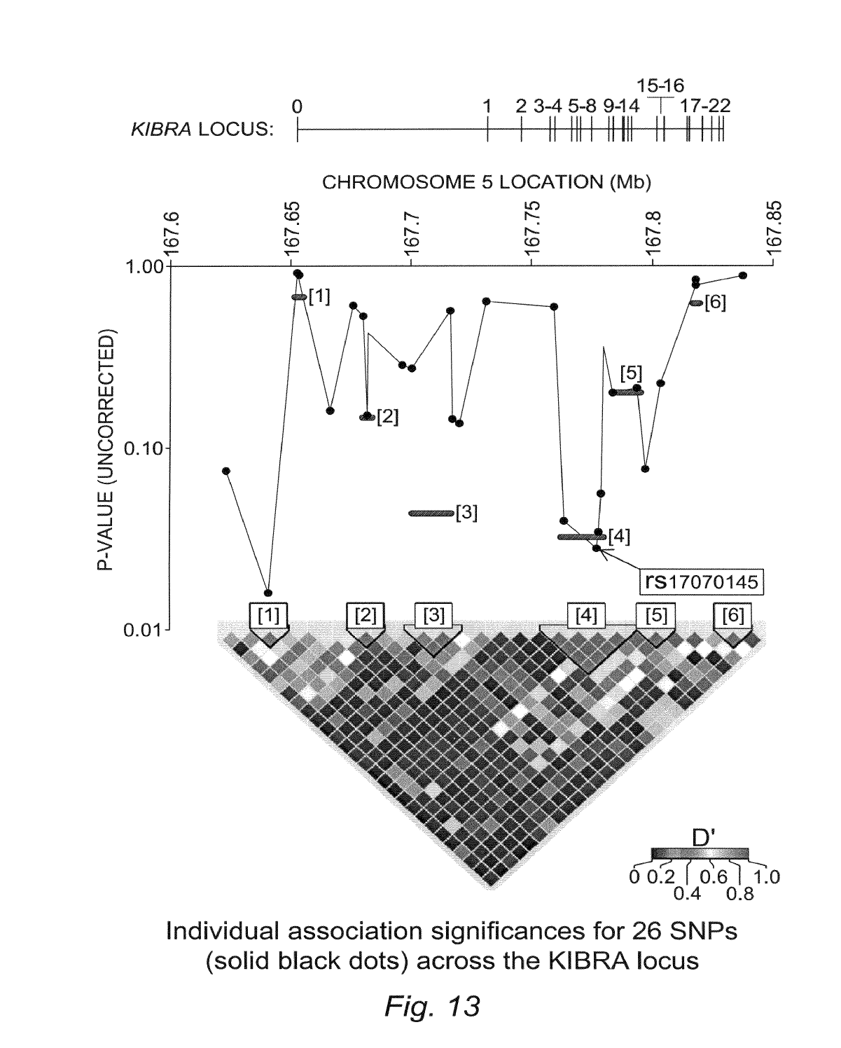

Fine-mapping the genomic region harboring KIBRA and the flanking genes RARS and ODZ2 with 19 additional SNPs was performed to ensure that the observed association of KIBRA SNP rs17070145 with episodic memory was not due to linkage disequilibrium (LD) with genetic variations in nearby genes. Three haplotype blocks were observed within KIBRA (FIG. 13). SNP rs17070145 and the corresponding haplotype block (block 2) yielded the highest significance levels (P=0.000004 and P=0.00001, respectively), which remained significant after Bonferroni correction for 40 comparisons (twenty SNPs analyzed with both the additive and the dominant model, P=0.00016 and P=0.0004, respectively). Accordingly, the observed association is unrelated to LD with adjacent genes.

### Example 8

### Investigation of KIBRA Genotype to Memory-Related Brain Functions with fMRI

The relation of the KIBRA genotype to memory-related brain fimctions was investigated with fimctional magnetic resonance imaging (fMRI). fMRI measures regional changes in deoxyhemoglobin levels, a marker of local neuronal activity. Thirty subjects from the Swiss population (15 carriers of the rs17070145*Tallele versus 15 non-carriers) underwent SRI. The allelic groups were matched for sex (5 males and 10 females in each group), education (P=0.7), age (P=0.8) and the His452Tyr genotype of the 5-HT2a receptor gene (P=0.4) because these variables have been shown to influence memory (Degonda, N. et al., Neuron 46, 505 (2005)). In order to avoid genotype-unrelated performance effects on brain activations and to instead capture genotype-dependent differences in brain activation patterns, the groups were matched for 5-min delayed recall performance (P=1.0). It was therefore expected that non-carriers of the T allele need more activations in memory-related brain regions to reach the same level of memory performance (Henke, K. et al.Proc. Natl. Acad. Sci. U. S. A 96, 5884 (1999)). Furthermore, because KIBRA was associated with human episodic memory which depends on the function of the hippocampus (Squire, L. R. & Alvarez, P. Curr. Opin. Neurobiol. 5, 169 (1995); Buther, K. et al. Biochem. Biophys. Res. Commun. 317, 703 (2004)) and because KIBRA is expressed in the hippocampus, it was hypothesized that KIBRA genotypes might affect episodic memory-related information processing in the human hippocampus. As neuroimaging studies have found that the hippocampus is especially activated by associative episodic memory tasks (Drier, E. A. et al., Nat. Neurosci. 5, 316 (2002); Sacktor, T. C. et al., Proc. Natl. Acad. Sci. U. S. A 90, 8342 (1993)), the impact of the KIBRA genotype on hippocampal activations in a face-profession associative task was tested (Drier, E. A. et al., Nat. Neurosci. 5, 316 (2002)).

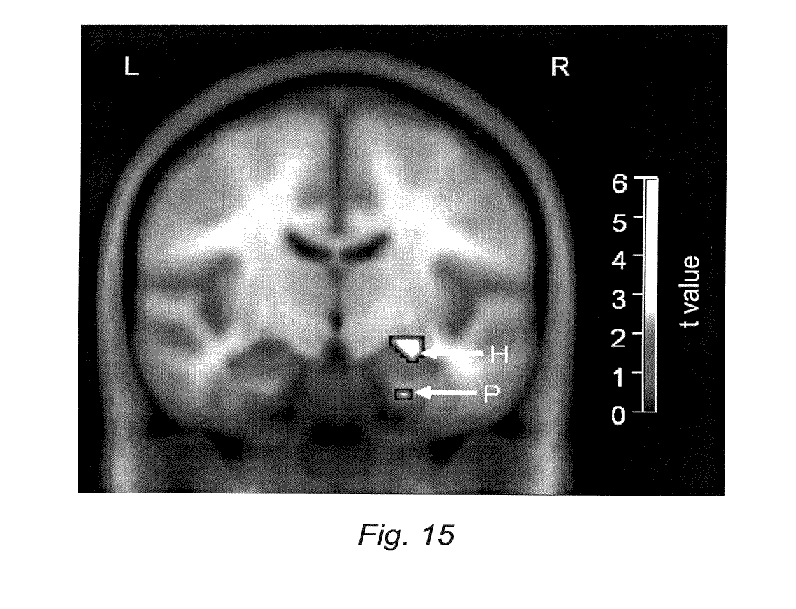

As expected based on the matching, there were no allele-dependent differences in fMRI retrieval performance (P=0.5). During memory retrieval, non-carriers of the T allele showed significantly increased brain activations compared to T allele carriers in the medial temporal lobe (local maximum in the right hippocampus at coordinate position [26, −12, −14], t=4.76, P<0.001, coordinates according to the Montreal Neurological Institute) (FIG. 15). Non-carriers of the T allele also showed increased activations in the frontal cortex (local maxima in the right medial frontal gyrus (Brodmann area 8/9) at coordinate position [30, 42, 42], t=4.24, P<0.001, in the left medial frontal gyrus (Brodmann area 6) at [−24, 10, 56], t=4.38, P<0.001), and in the parietal cortex (local maximum in the right inferior parietal lobulus (Brodmann area 40) at coordinate position [50, −24, 30], t=3.97, P<0.001). There were no additional increased brain activations in non-carriers of the T allele in this episodic memory task. Furthermore, in a working memory task non-carriers of the T allele failed to show any increased brain activations compared to T allele carriers, indicating that the above reported activations in non-carriers were specific to episodic memory retrieval.

All brain regions reported above belong to a network important for episodic memory retrieval (Ubach, J. Et al. EMBO J. 17, 3921 (1998)) which has also been activated during memory retrieval in the present study, including the medial temporal lobe (local maximum in the right hippocampus at coordinate position [32, −8, −24], t=4.27, P<0.001). The findings therefore indicate that non-carriers of the T allele need more activation in these memory retrieval-related brain regions to reach the same level of retrieval performance as T allele carriers. There were no greater task-related cortical activations in the T allele group as compared to non carriers of the T allele. No allele-dependent differences in brain activations during encoding were found, suggesting that the genotype did not affect episodic memory at this early stage of memory formation. In an additional working memory task, no allele-dependent differences in brain activation in these regions were seen, indicating that the above reported activations in non-carriers were specific to episodic memory retrieval. Automated voxel-based algorithms (SPM2) (Cabeza, R. & Nyberg, L. J. Cogn Neurosci. 12, 1 (2000)) and manual volume measurements failed to reveal significant allele-dependent differences in volumes of the hippocampus or the parahippocampal gyrus, or in white and grey matter volumes, suggesting that functional imaging results were not biased by morphological differences. Furthermore, there were no significant correlations between memory measures and any of the brain volumes.

In addition to the SNP-based analysis, fMRI analysis based on the memory associated KIBRA haplotype revealed a similar genetic influence on hippocampal activation during memory retrieval (local maximum at coordinate position [24, −10, −14], t=5.24, P<0.001). An identical fRI activation was seen in carries of the CAMTAL risk allele.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method for improving memory and learning in a subject, the method comprising:
administering to a patient in need thereof, a therapeutically effective amount of a compound of Formula I: 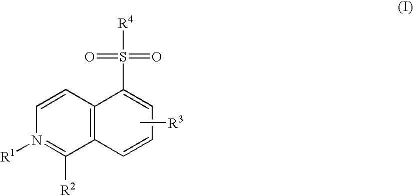
wherein
R¹ is absent or is a member selected from the group consisting of hydrogen and C₁₋₆alkyl;
R² is a member selected from the group consisting of hydrogen, hydroxy and halogen;
R³ is a member selected from the group consisting of hydrogen and C₁₋₆alkyl;
R⁴ is an N-linked heterocyclic ring system having from 5 to 8 ring members and two N ring heteroatoms, substituted with 0-3 R⁵ groups, wherein each R⁵ is independently a member selected from the group consisting of hydrogen, C₁₋₆alkyl, benzyl and phenyl;
and prodrugs, salts, hydrates and solvates thereof.

2. The method of claim 1, wherein R⁴ is a 7-membered heterocyclic ring system.

3. The method of claim 2, wherein the compound is of Formula Ia:

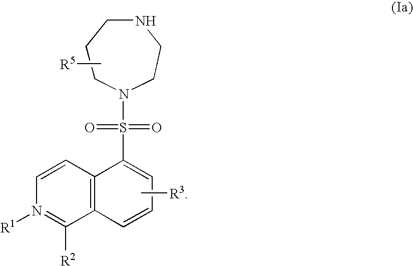

4. The method of claim 3, wherein the compound is:

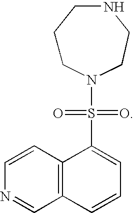

5. The method of claim 3, wherein the compound is:

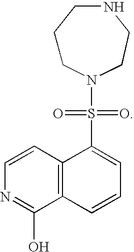

6. The method of claim 3, wherein the compound is the HCl salt.

7. The method of claim 6, wherein the compound is:

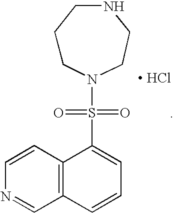

8. The method of claim 6, wherein the compound is:

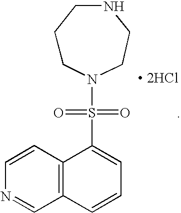

9. The method of claim 6, wherein the compound is the hydrate.

10. The method of claim 9, wherein the compound is: 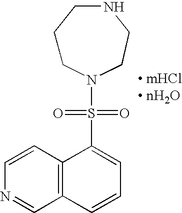
wherein
m is 1 or2;and
n is from ½ to 3.

11. The method of claim 1, wherein the compound of Formula I is administered with a nitric oxide enhancing agent.

12. The method of claim 11, wherein the nitric oxide enhancer is selected from the group consisting of a PDE5 inhibitor, a nitric oxide donor molecules, or a HMG Co A Reductase.

13. The method of claim 12, wherein the nitric oxide enhancer is selected from the group consisting of Sildenafil, Tadalafil, Vardenafil, sodium nitroprusside, nitroglycerin, Atorvastatin, Simvastatin, Lovastatin, Fluvastatin, Pravastatin, Mevastatin, Pitavastatin, and Rosuvastatin.

14. The method of claim 11, wherein the compound of Formula I and the nitric oxide enhancing agent are administered in together in the same composition.

15. The method of claim 11, wherein the compound of Formula I and the nitric oxide enhancing agent are administered in together different compositions.

16. The method of claim 15, wherein the compound of Formula I and the nitric oxide enhancing agent are administered at the same time.

17. The method of claim 15, wherein the compound of Formula I and the nitric oxide enhancing agent are administered at different times.

18. A method for improving memory and learning in a subject, the method comprising:
administering to a patient in need thereof, a therapeutically effective amount of a compound of Formula II: 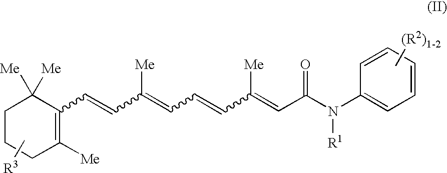
wherein
R¹ is a member selected from the group consisting of hydrogen and C₁₋₆ alkyl;
each R² is a member selected from the group consisting of hydrogen, C₁₋₆ alkyl, hydroxy and —O—C₁₋₆ alkyl;
R³ is a member selected from the group consisting of hydrogen and C₁₋₆ alkyl; each [symbol] represents that the double bond to which it is attached is cis or trans; and prodrugs, salts, hydrates and solvates thereof.

19. The method of claim 18, wherein the compound is of Formula IIa:

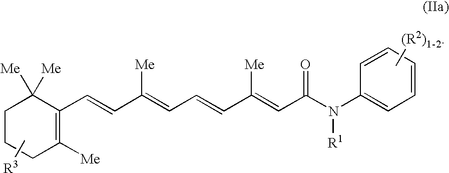

20. The method of claim 19, wherein the compound is:

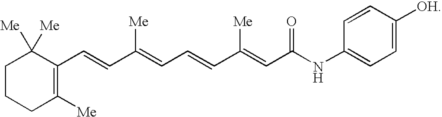

21. A method for improving memory and learning, the method comprising:
administering to a patient in need thereof, a therapeutically effective amount of a compound of Formula III:
(R¹)ₓ—C—B-Tyr-Arg-Arg-A-A-Arg-Arg-Trp-Arg-Lys-B—(R²)_{y} and conservatively modified variations thereof, in which:
R¹ is an amino acid sequence comprising from 1 to about 40 amino acids wherein each amino acid is independently selected from the group consisting of naturally occurring amino acids and amino acid analogs;
R² is an amino acid sequence comprising from 1 to about 40 amino acids wherein each amino acid is independently selected from the group consisting of naturally occurring amino acids and amino acid analogs; A represents glycine or alanine; B represents isoleucine, leucine, methionine or valine; C represents serine or threonine; and
x and y are independently selected and are equal to zero or one.

22. The method of claim 21 comprising:
administering to a patient in need thereof, a therapeutically effective amount of a compound of Formula III:
(R¹)ₓ-Ser-Ile-Tyr-Arg-Arg-Gly-Ala-Arg-Arg-Trp-Arg-Lys-Leu —(R²)_{y} and conservatively modified variations thereof, in which:
R¹ is an amino acid sequence comprising from 1 to about 40 amino acids wherein each amino acid is independently selected from the group consisting of naturally occurring amino acids and amino acid analogs;
R² is an amino acid sequence comprising from 1 to about 40 amino acids wherein each amino acid is independently selected from the group consisting of naturally occurring amino acids and amino acid analogs; A represents glycine or alanine; B represents isoleucine, leucine, methionine or valine; C represents serine or threonine; and
x and y are independently selected and are equal to zero or one.

23. The method of claim 21 comprising:
administering to a patient in need thereof, a therapeutically effective amount of a compound of Formula III:
(R¹)ₓ-Ser-Ile-Tyr-Arg-Arg-Gly-Ala-Arg-Arg-Trp-Arg-Lys-Leu —(R²)_{y} and conservatively modified variations thereof, in which:
x and y are zero.

24. A method of identifying an increased risk of developing Alzheimer's disease in a subject, the method comprising the steps of obtaining a biologicial sample from a subject and identifying the presence or absence of the C-allele of SNP rs17070145 in nucleic acid from the sample, wherein the presence of one or more copies of the C-allele indicates an increased risk in developing Alzheimer's disease as compared to subjects lacking the C-allele.

25. The method of claim 24, wherein the sample is blood.

26. The method of claim 24, wherein the nucleic acid is DNA.

27. The method of claim 24, wherein the allele is identified using PCR.
